(19) United States
(12) Patent Application Publication
(10) Pub. No.: US 2022/0228146 A1
(43) Pub. Date: Jul. 21, 2022

(54) MICRO-RNA SITE BLOCKING OLIGONUCLEOTIDES FOR THE TREATMENT OF EPILEPTIC ENCEPHALOPATHY AND NEURODEVELOPMENTAL DISORDERS

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Beverly DAVIDSON, Philadelphia, PA (US); Benjamin L. PROSSER, Philadelphia, PA (US); Ingo HELBIG, Philadelphia, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 17/607,837

(22) PCT Filed: May 6, 2020
(86) PCT No.: PCT/US2020/031672

§ 371 Date: Oct. 29, 2021

Related U.S. Application Data
(60) Provisional application No. 62/875,308, filed on Jul. 17, 2019.
(60) Provisional application No. 62/843,859, filed on May 6, 2019.

(51) Int. Cl. C12N 15/113

(52) U.S. Cl. CPC C12N 15/113; C12N 2310/20; C12N 2310/11

(57) ABSTRACT

Disclosed herein are compounds and methods for modulating expression of disease-causing genes such as STXBP1, SCN1A, SCN2A, SCN8A, SLC6A1, and MECP2. Such compounds and methods are useful to treat, prevent, or ameliorate epileptic encephalopathy and neurodevelopmental disorders in an individual in need thereof.

REFERENCE TO RELATED APPLICATIONS

The present application claims the priority benefit of U.S. provisional application No. 62/875,308, filed Jul. 17, 2019, and U.S. provisional application No. 62/843,859, filed May 6, 2019, the entire contents of each of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on May 4, 2020, is named CHOPP0029WO_ST25.txt and is 12.7 kilobytes in size.

### BACKGROUND

### I. Field

The present disclosure relates to the fields of medicine, genetics, and molecular biology. Provided are antisense compounds that target miRNAs involved with diseases such as epileptic encephalopathy and neurodevelopmental disorders. More specifically, methods related to upregulating gene expression of STXBP1, SCN1A, SCN2A, SCN8A, SLC6A1, MECP2 and other causative genes using site blocking antisense oligonucleotides (SBO's) that compete for binding with miRNAs.

### II. Related Art

Targeting disease-causing gene sequences was first suggested more than thirty years ago (Belikova et al., Tet. Lett., 1967, 37, 3557-3562), and antisense activity was demonstrated in cell culture more than a decade later (Zamecnik et al., Proc. Natl. Acad. Sci. U.S.A., 1978, 75, 280-284). One advantage of antisense technology in the treatment of a disease or condition that stems from a disease-causing gene is that it is a direct genetic approach that has the ability to modulate (increase or decrease) the expression of specific disease-causing genes. Another advantage is that validation of a therapeutic target using antisense compounds results in direct and immediate discovery of the drug candidate; the antisense compound is the potential therapeutic agent.

Generally, the principle behind antisense technology is that an antisense compound hybridizes to a target nucleic acid and modulates gene expression, activity, or function. The modulation of gene expression can be achieved by, for example, target degradation or occupancy-based inhibition. Antisense technology is an effective means for modulating the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA.

### SUMMARY

Thus, in accordance with the present disclosure, there is provided a compound comprising a single-stranded oligonucleotide, wherein the single-stranded oligonucleotide consists of 13 to 30 linked nucleosides and has a nucleobase sequence comprising a complementary region having at least 7 contiguous nucleobases complementary to an equal-length portion within a target region of a target nucleic acid, wherein the target nucleic acid is located in an mRNA transcript selected from the group consisting of STXBP1, SCN1A, SCN2A, SCN8A, SLC6A1, and MECP2.

The complementary region may comprise at least 10, 12, 14, 16, 18, 20 contiguous nucleobases complementary to an equal-length portion within the target region of the mRNA transcript. The target region may at least partially overlaps a binding site for miR218, miR148b, miR30b, miR30c, miR424, miR15b, miR3911, miR338, or miR942. The target region may comprises an at least 10, 12, 14, 16, 18, or 20 continguous nucleotide segment of any one of: UCACCCCACAGAAACUGCUGGACACACUGAAGAAACU (SEQ ID NO: 1), UGAGCACACCAUUUGUGCUGCUGCUGUUGUCGUGAAAU (SEQ ID NO: 2), GUUUGAAAGUACUGAAGCACAAACAUAUAUCAUCUCU (SEQ ID NO: 3), GUCUGUCUUGAAACUUGUUUACCUUAAAAUUAUCAGAA (SEQ ID NO: 4), UUUGAAAUCUCCCCUUGCACUGAGAUUAGUCGUCAGA (SEQ ID NO: 5), or CCAAAGAAACAAAGAUCCACACACACUCCUCACCCCAC (SEQ ID NO: 6). The target region may be a sequence selected from CACACATCCTCACCCCACAG (SEQ ID NO: 7), ACCCCACAGAAACTGCTGGA (SEQ ID NO: 8), CAGAAACTGCTGGACACACT (SEQ ID NO: 9), CCATTTGTGCTGCTGCTGTT (SEQ ID NO: 10), TTGTGCTGCTGCTGTTGTCG (SEQ ID NO: 11), CTGCTGCTGTTGTCGTGAAA (SEQ ID NO: 12), GAAAGTACTGAAGCACAAAC (SEQ ID NO: 13), GCACAAACATATATCATCTC (SEQ ID NO: 14), ATCATCTCTGTACCATTCTG (SEQ ID NO: 15), GUCUGUCUUGAAACUUGUUU (SEQ ID NO: 16), UCUUGAAACUUGUUUACCUU (SEQ ID NO: 17), AAACUUGUUUACCUUAAAAU (SEQ ID NO: 18), UGUUUACCUUAAAAUUAUCA (SEQ ID NO: 19), UUUGAAAUCUCCCCUUGCAC (SEQ ID NO: 20), AAUCUCCCCUUGCACUGAGA (SEQ ID NO: 21), CCCCUUGCACUGAGAUUAGU (SEQ ID NO: 22), UGCACUGAGAUUAGUCGUCA (SEQ ID NO: 23), CCAAAGAAACAAAGAUCCAC (SEQ ID NO: 24), GAAACAAAGAUCCACACACA (SEQ ID NO: 25), AAAGAUCCACACACACUCCU (SEQ ID NO: 26), or UCCACACACACUCCUCACCC (SEQ ID NO: 27).

The compound nucleobase sequence may comprise a sequence that is at least 70% identical to a complement of an at least 10, 12, 14, 16, 18, or 20 continguous nucleotide segment of any one of: UCACCCCACAGAAACUGCUGGACACACUGAAGAAACU (SEQ ID NO: 1), UGAGCACACCAUUUGUGCUGCUGCUGUUGUCGUGAAAU (SEQ ID NO: 2), GUUUGAAAGUACUGAAGCACAAACAUAUAUCAUCUCU (SEQ ID NO: 3), GUCUGUCUUGAAACUUGUUUACCUUAAAAUUAUCAGAA (SEQ ID NO: 4), UUUGAAAUCUCCCCUUGCACUGAGAUUAGUCGUCAGA (SEQ ID NO: 5), or CCAAAGAAACAAAGAUCCACACACACUCCUCACCCCAC (SEQ ID NO: 6). The compound DNA sequence may be CTGTGGGGTGAGGATGTGTG (SEQ ID NO: 28), TCCAGCAGTTTCTGTGGGGT (SEQ ID NO: 29), AGTGTGTCCAGCAGTTTCTG (SEQ ID NO: 30), AACAGCAGCAGCACAAATGG (SEQ ID NO: 31), CGACAACAGCAGCAGCACAA (SEQ ID NO: 32), TTTCACGACAACAGCAGCAG (SEQ ID NO: 33), GTTTGTGCTTCAGTACTTTC (SEQ ID NO: 34), GAGATGATATATGTTTGTGC (SEQ ID NO: 35), CAGAATGGTACAGAGATGAT (SEQ ID NO: 36), AAACAAGTTTCAAGACAGAC (SEQ ID NO: 37), AAGGTAAACAAGTTTCAAGA (SEQ ID NO: 38), ATTTTAAGGTAAACAAGTTT (SEQ ID NO: 39), TGATAATTTTAAGGTAAACA (SEQ ID NO: 40), GTGCAAGGGGAGATTTCAAA (SEQ ID NO: 41), TCTCAGTGCAAGGGGAGATT (SEQ ID NO: 42), ACTAATCTCAGTGCAAGGGG (SEQ ID NO: 43), TGACGACTAATCTCAGTGCA (SEQ ID NO: 44), GTGGATCTTTGTTTCTTTGG (SEQ ID NO: 45), TGTGTGTGGATCTTTGTTTC (SEQ ID NO: 46), AGGAGTGTGTGTGGATCTTT (SEQ ID NO: 47), or GGGTGAGGAGTGTGTGTGGA (SEQ ID NO: 48). The compound RNA sequence may be CUGUGGGGUGAGGAUGUGUG (SEQ ID NO: 49), UCCAGCAGUUUCUGUGGGGU (SEQ ID NO: 50), AGUGUGUCCAGCAGUUUCUG (SEQ ID NO: 51), AACAGCAGCAGCACAAAUGG (SEQ ID NO: 52), CGACAACAGCAGCAGCACAA (SEQ ID NO: 32), UUUCACGACAACAGCAGCAG (SEQ ID NO: 53), GUUUGUGCUUCAGUACUUUC (SEQ ID NO: 54), GAGAUGAUAUAUGUUUGUGC (SEQ ID NO: 55), CAGAAUGGUACAGAGAUGAU(SEQ ID NO: 56), AAACAAGUUUCAAGACAGAC (SEQ ID NO: 57), AAGGUAAACAAGUUUCAAGA (SEQ ID NO: 58), AUUUUAAGGUAAACAAGUUU (SEQ ID NO: 59), UGAUAAUUUUAAGGUAAACA (SEQ ID NO: 60), GUGCAAGGGGAGAUUUCAAA (SEQ ID NO: 61), UCUCAGUGCAAGGGGAGAUU (SEQ ID NO: 62), ACUAAUCUCAGUGCAAGGGG (SEQ ID NO: 63), UGACGACUAAUCUCAGUGCA (SEQ ID NO: 64), GUGGAUCUUUGUUUCUUUGG (SEQ ID NO: 65), UGUGUGUGGAUCUUUGUUUC (SEQ ID NO: 66), AGGAGUGUGUGUGGAUCUUU (SEQ ID NO: 67), or GGGUGAGGAGUGUGUGUGGA (SEQ ID NO: 68).

The single-stranded oligonucleotide may comprise a terminal group at the 5′-end of the oligonucleotide and the 5′-terminal nucleoside and terminal group of the compound may have Formula I:

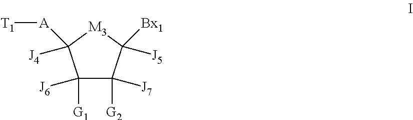

wherein:
T₁ is a phosphorus moiety;
A has a formula selected from among:

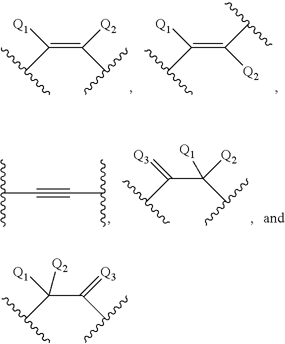

Q₁ and Q₂ are each independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl, and N(R₃)(R₄); Q₃ is selected from among: O, S, N(R₅), and C(R₆)(R₇); each R₃, R₄ R₅, R₆ and R₇ is independently selected from among: H, C₁-C₆ alkyl, substituted C₁-C₆ alkyl, and C₁-C₆ alkoxy; M₃ is selected from among: O, S, NR₁₄, C(R₁₅)(R₁₆), C(R₁₅)(R₁₆)C(R₁₇)(R₁₈), C(R₁₅)═C(R₁₇), OC(R₁₅)(R₁₆), and OC(R₁₅)(Bx₂); R₁₄ is selected from among: H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl; R₁₅, R₁₆, R₁₇ and R₁₈ are each independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl; if Bx₂ is present, then Bx₂ is a nucleobase and Bx₁ is selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl; if Bx₂ is not present, then Bx₁ is a nucleobase; either each of J₄, J₅, J₆ and J₇ is independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl; or J₄ forms a bridge with one of J₅ or J₇ wherein the bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl; each R₁₉, l R₂₀ and R₂₁ is independently selected from among: H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl; one of G₁ and G₂ is selected from among: H, OH, halogen and O—[C(R₈)(R₉)]ₙ—[(C═O)ₘ—X₁]ⱼ—Z; and the other of G₁ and G₂ is: O-T₂; T₂ is an internucleoside linking group linking the 5′-terminal nucleoside of Formula I to the remainder of the oligonucleotide; each R₈ and R₉ is independently selected from among: H, halogen, C₁-C₆ alkyl, and substituted C₁-C₆ alkyl; X₁ is O, S or N(E₁); Z is selected from among: H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, and N(E₂)(E₃); E₁, E₂ and E₃ are each independently selected from among: H, C₁-C₆ alkyl, and substituted C₁-C₆ alkyl; n is from 1 to 6; m is 0 or 1; j is 0 or 1; provided that, if j is 1, then Z is other than halogen or N(E₂)(E₃); each substituted group comprises one or more optionally protected substituent groups independently selected from among: a halogen, OJ₁, N(J₁)(J₂), =NJ₁, SJ₁, N₃, CN, OC(═X₂)J₁, OC(═X₂)N(J₁)(J₂), and C(═X₂)N(J₁)(J₂); X₂ is O, S or NJ₃; and each J₁, J₂ and J₃ is independently selected from among: H and C₁-C₆ alkyl.

Further, M₃ may be selected from among O, CH═CH, OCH₂, and OC(H)(Bx₂). Each of J₄, J₅, J₆ and J₇ may be H. J₄ may form a bridge with either J₅ or J₇. A may have the formula:

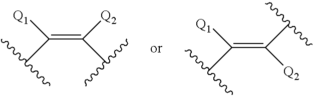

wherein Q₁ and Q₂ are each independently selected from among H, halogen, C₁-C₆ alkyl, substituted C₁-C₆ alkyl, C₁-C₆ alkoxy, and substituted C₁-C₆ alkoxy. Each of Q₁ and Q₂ may be H. Q₁ and Q₂ may be each independently selected from among H and a halogen. One of Q₁ and Q₂ may be H and the other of Q₁ and Q₂ may be F, CH₃ or OCH₃.

T₁ may have the formula:

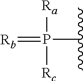

wherein Rₐ and R_{c} are each independently selected from among hydroxyl, protected hydroxyl, thiol, protected thiol, C₁-C₆ alkyl, substituted C₁-C₆ alkyl, C₁-C₆ alkoxy, substituted C₁-C₆ alkoxy, amino, protected amino or substituted amino; and R_{b} is O or S. R_{b} may be O and Rₐ and R_{c} may each be independently selected from among: OH, OCH₃, OCH₂CH₃, OCH(CH₃)₂.

One of G₁ and G₂ is selected from among a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁₀)(R₁₁), O(CH₂)₂—ON(R₁₀)(R₁₁), O(CH₂)₂—O(CH₂)₂—N(R₁₀)(R₁₁), OCH₂C(═O)—N(R₁₀)(R₁₁), OCH₂C(═O)—N(R₁₂)—(CH₂)₂—N(R₁₀)(R₁₁), and O(CH₂)₂—N(R₁₂)—C(═NR₁₃)[N(R₁₀)(R₁₁)]; wherein R₁₀, R₁₁, R₁₂ and R₁₃ are each, independently, H or C₁-C₆ alkyl. G₁ and G₂ may be selected from among a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂. G₁ and G₂ may be selected from among F, OCH₃, and O(CH₂)₂—OCH₃. One of G₁ and G₂ may be O(CH₂)₂—OCH₃.

The 5′-terminal nucleoside and terminal group of the compound may have the Formula III:

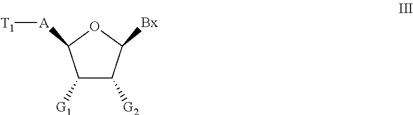

wherein A has the formula:

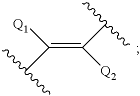

wherein Q₁ and Q₂ may be each independently selected from among: H a halogen, C₁-C₆ alkyl, substituted C₁-C₆ alkyl, C₁-C₆ alkoxy, and substituted C₁-C₆ alkoxy. Q₁ and Q₂ may each be independently selected from among: H, F, CH₃, and OCH₃. The 5′-terminal nucleoside and the terminal group may have Formula V:

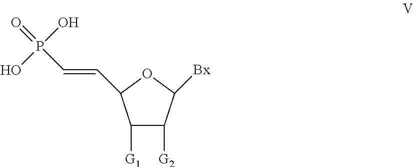

wherein Bx may be selected from among uracil, thymine, cytosine, 5-methyl cytosine, adenine, and guanine; one of G₁ and G₂ is selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂ and OCH₂—N(H)—C(═NH)NH₂; and the other of G₁ and G₂ may be O-T₂, wherein T₂ is a phosphorothioate internucleoside linking group linking the compound of Formula V to the remainder of the oligonucleotide.

The single-stranded oligonucleotide comprises at least two modified sugar moieties. Each modified sugar moiety may be independently selected from among: 2′-F, 2′-MOE, 2′-OMe, LNA, F-HNA, and cEt. The modified oligonucleotide may be fully modified, such as with 2′-OMe, optionally further comprising a phosphorothioate backbone. The modified oligonucleotide may comprise at least one region having sugar motif:

-[(A)ₓ-(B)_{y}-(A)_{z}]_{q}-

wherein A is a modified nucleoside of a first type, B is a modified nucleoside of a second type; each x and each y may be independently 1 or 2; z is 0 or 1; and q is 3-15. Each x and each y may be 1. A may be a modified nucleoside selected from among a 2′-F, a 2′-OMe, and a F-HNA modified nucleoside. B may be a modified nucleoside selected from among a 2′-F, a 2′-OMe, and a F-HNA modified nucleoside. A may be a 2′-F modified nucleoside and B may be a 2′-OMe modified nucleoside. B may be a 2′-F modified nucleoside and A may be a 2′-OMe modified nucleoside.

The modified oligonucleotide may comprise 1-4 3′-terminal nucleosides, each comprising the same modified sugar moiety, wherein the modified sugar moiety of the 1-4 3′-terminal nucleosides is different from the modified sugar moiety of the immediately adjacent nucleoside. The 3′-terminal nucleosides may each be 2′-MOE nucleosides. The compound may comprise two 3′-terminal nucleosides.

The modified oligonucleotide may comprise at least one modified internucleoside linkage. Each internucleoside linkage of the modified oligonucleotide is selected from a phosphorothioate internucleoside linkage and an unmodified, phosphate internucleoside linkage. Each of the 6-10 3′-most internucleoside linkages of the modified oligonucleotide may be a phosphorothioate internucleoside linkage. The 5′-most internucleoside linkage of the modified oligonucleotide may be a phosphorothioate internucleoside linkage. The modified oligonucleotide may comprise a region of at least 4 internucleoside linkages that alternate between phosphorothioate and phosphate internucleoside linkages.

The single-stranded oligonucleotide may have two mismatches relative to the target region of the mRNA transcript, three mismatches relative to the target region of the mRNA transcript, four mismatches relative to the target region of the mRNA transcript, five mismatches relative to the target region of the mRNA transcript, or six mismatches relative to the target region of the mRNA transcript. The 5′-most nucleobase and 3′-most nucleobase of the single-stranded oligonucleotide may be mismatches relative to the target region of the mRNA transcript. The 5′-most nucleobase and the two 3′-most nucleobases of the single-stranded oligonucleotide may be mismatches relative to the target region of the mRNA transcript. The nucleobases at positions 9 and 14 of the single-stranded oligonucleotide may be mismatches relative to the target region of the mRNA transcript. The nucleobases at positions 9, 10, and 11 of the single-stranded oligonucleotide may be mismatches relative to the target region of the mRNA transcript.

The complementary region may be between the 5′-most nucleoside and the two 3′-most nucleosides of the single-stranded oligonucleotide and may be 100% complementary to the target region of the mRNA transcript. The complementary region may be between the 5′-most nucleoside and the two 3′-most nucleosides of the single-stranded oligonucleotide and may be 90% complementary to the target region of the mRNA transcript. The complementary region may be between the 5′-most nucleoside and the two 3′-most nucleosides of the single-stranded oligonucleotide and may be 80% complementary to the target region of the mRNA transcript.

Each of the nucleobases in the complementary region of the single-stranded oligonucleotide may be selected from among adenine, guanine, cytosine, 5′-methylcytosine, thymine, and uracil.

Tach of the nucleobases in the single-stranded oligonucleotide may be selected from among adenine, guanine, cytosine, 5′-methylcytosine, thymine, and uracil.

The single-stranded oligonucleotide may not comprise a modified nucleobase. Alternatively, the single-stranded oligonucleotide may comprise 5-methylcytosine.

The phosphorus moiety may be an unmodified phosphate. The phosphorus moiety may be a 5′-(E)-vinylphosphonate group having the formula:

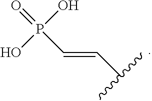 [SMILES: */C=C/P(=O)(O)O]

The compound may comprise an unlocked nucleic acid or an abasic nucleoside. The nucleobase attached to the sugar moiety of the unlocked nucleic acid may be mismatched relative to the corresponding nucleobase of the target region of the mRNA transcript.

The compound may consist essentially of the single-stranded oligonucleotide or consist of the single-stranded oligonucleotide.

In another embodiment, there is provided a pharmaceutical composition comprising the compound described above dispersed in a pharmaceutically acceptable carrier or diluent.

In yet another embodiment, there is provided a method of treating epileptic encephalopathy or a neurodevelopmental disorder comprising contacting a cell with the compound or composition as described above. The cell may be in vitro, followed be introduction of the cell into the subject. The cell may be in vivo, such as is in an animal, such as a human. Contacting may comprise delivery into the cerebrospinal fluid (CFS). Contacting may comprise multiple administration of the compound or composition. The method may further comprise assessing expression of a target protein in CSF of said animal following contacting. The animal or human may be an infant or child. The expression of a protein encoded by a target mRNA may be increased approximately 2-fold as compared to pretreatment levels.

Also provided are a use of the compound or composition described above for the manufacture of a medicament for treating epileptic encephalopathy or a neurodevelopmental disorder, and a use of the compound or composition as described above for treating epileptic encephalopathy or a neurodevelopmental disorder.

As used herein in the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. STXBP1 mRNA and protein levels from a neuronal cell line (SH-SY5Y) transfected with antisense oligonucleotides to inhibit miR-218. Disrupting miR-218 relieves the inhibition of STXBP1, resulting in a ˜2-fold increase in protein expression. Each data point represents one independent biological replicate. Each comparison is statistically significant at p<0.01.

FIGS. 2A-D. (FIG. 2A) STXBP1 protein levels (via western blot) after transfection of rat glioblastoma line (PC12 cells) with ASO to inhibit miR-218 and miR-424 for 48 hrs. (FIG. 2B) mRNA (from qPCR) and (FIG. 2C) protein levels of STXBP1 after transfection of human neuroblastoma line (SH-SY5Y cells) for 48 hrs. (FIG. 2D) STXBP1 protein levels (via western blot) following lentiviral transduction of ASO against miR-218 in SH-SY5Y cells for 48 hrs.

FIG. 3. Nine ASOs under testing. Three ASOs each covering three different miRNA binding sites on the 3′UTR of STXBP1. The miRNA binding site is shown in the second column (SEQ ID NOs: 7-15, listed top to bottom), with the SBO Seq DNA sequence in the second to last column (SEQ ID NOs: 28-36, listed top to bottom), and with the ASO sequence in the final column (SEQ ID NOs: 49-52, 38, and 53-56, listed top to bottom). The nine ASOs (SEQ ID NOs: 49-52, 38, and 53-56, listed top to bottom) are also listed with the m and * denoting the modifications to each nucleotide.

FIGS. 4A-B. (FIG. 4A) CRISPR/Cas9 mediated excision of the miR-218 gene leads to an upregulation in protein expression of miR-218 targets STXBP1 and SCN1A in a human neuronal cell line, as shown via western blotting. ACTB used as loading control. (FIG. 4B) Inhibition of miR-218 via Cas9-mediated excision upregulates STXBP1 and SCN1A in human neuronal cells. All box plots depict 25-75% CI, with mean line+/−1SD, *p<0.05.

FIGS. 5A-D. (FIG. 5A&B) Luciferase Reporter tool to screen ASOs that upregulate STXBP1 3′UTR. (FIG. 5A) Plasmid map of reporter screening tool. The 3′UTR of STXBP1 is cloned downstream of a luciferase reporter gene. If an ASO disrupts miR-mediated repression of STXBP1 by binding to its 3′UTR, this results in increased light emitted from the sample. This tool, when expressed in a stable SH-SY5Y cell line, can be used for high-throughput screening of ASOs to upregulate target genes. (FIG. 5B) Results of reporter assay upon inhibition of miR-218 and miR-424. Luminescence (and thus gene expression) is increased when cells expressing this reporter are transduced with lentivirus encoding inhibitors of miR-218 or miR-424, but not when transduced with a scramble encoding lentivirus or in the absence of the 3′UTR of STXBP1. (FIG. 5C) Schematic of miR-reporter assay and controls. (FIG. 5D) Reporter construct transfected into SH-SY5Y cells reveals increased STXBP1 3′UTR expression upon inhibition of miR-218 and miR-424. All box plots depict 25-75% CI, with mean line+/−1SD, *p<0.05.

FIG. 6. Left, schematic depicting miR-218 (SEQ ID NO: 69) binding site in the 3′UTR of STXBP1 (nucleotides 4-37 of SEQ ID NO: 3), and target sites of SBOs 7, 8, and 9. A similar 3 SBO strategy was used to flank the miR-424 and 338 sites on STXBP1 which are toward the 5′ end of the 3′ UTR. Right, results of triplicate experiments treating SH-SY5Y cells with 10 pmol/well of 9 SBOs that target the 3 putative miR-binding sites. All box plots depict 25-75% CI, with mean line+/−1SD, *p<0.05.

### DETAILED DESCRIPTION

In 2001, several groups used a novel cloning method to isolate and identify a large group of “microRNAs” (miRNAs) from C. elegans, Drosophila, and humans. Several hundreds of miRNAs have been identified in plants and animals—including humans—which do not appear to have endogenous siRNAs. Thus, while similar to siRNAs, miRNAs are nonetheless distinct. miRNAs thus far observed have been approximately 21-22 nucleotides in length and they arise from longer precursors, which are transcribed from non-protein-encoding genes. The precursors form structures that fold back on each other in self-complementary regions; they are then processed by the nuclease Dicer in animals or DCL1 in plants. miRNA molecules interrupt translation through precise or imprecise base-pairing with their targets.

The first miRNAs were identified as regulators of developmental timing in C. elegans, suggesting that miRNAs, in general, might play decisive regulatory roles in transitions between different developmental states by switching off specific targets. However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression.

The inventors describe here a method to upregulate STXBP1, SCN1A, SCN2A, SCN8A, SLC6A1, MECP2 and other causative genes for epileptic encephalopathies and neurodevelopmental disorders by blocking the ability of miRNAs to bind to and repress expression of the mRNA encoding this protein. Blocking this interaction serves as a therapeutic approach to treat childhood epilepsy and related neurodevelopmental disorders.

Pathogenic variants in each of these genes are known to be causative for epileptic encephalopathy and neurodevelopmental disorders, and this is commonly thought to occur via a haploinsufficiency mechanism. Thus, upregulation of the target gene should serve as a therapeutic approach by restoring protein expression back towards the endogenous level. There is existing bioinformatic or wet lab evidence that each gene is under partial control of micro(mi)RNA-218, as well as other regulatory miRNAs. The inventors have found that inhibition of miRNA binding is sufficient to upregulate STXBP1 mRNA and protein expression. miRNA binding can be disrupted using Site-Blocking antisense Oligonucleotides (SBOs) that compete for binding the miRNA binding site of the 3′UTR of the target gene. This represents a highly specific approach to upregulate gene expression, as miR binding sites and flanking regions that the SBOs recognize are quite unique throughout the genome. Further, such antisense oligonucleotides can be delivered into the cerebral spinal fluid (CSF) and have been found to have a favorable safety profile in clinical trials to date.

Thus, the disclosure provides, in certain aspects, for the administration of SBOs into the CSF as a therapeutic approach to restore protein expression of STXBP1, SCN1A, SCN2A, SCN8A, SLC6A1, and MECP2 in epileptic encephalopathies and neurodevelopmental disorders arising from haploinsufficiency of these genes of interest. These and other features of the disclosure are described in detail below.

TABLE 1
Exemplary ASOs covering different miRNA binding sites on the 3′UTR of STXBP1. The miRNA
binding site is shown in the second column as underlined text, with the ASO sequence in
the final column.

| | STXBP1 Target | Target | | | | | | |
|---|---|---|---|---|---|---|---|---|
| | Region (5′-3′) | Sequence | Binding | | G + C | Free | SBO Sequence | SBO Sequence |
| miRNA | RNA | (5′-3′) | Site | Tm | % | Energy | (DNA, 5′-3′) | (RNA, 5′-3′) |
| miR491/ | UCACCCCACAGA | CACACATCCTCA | 23-42 | 57.9 | 60 | -1.6 | CTGTGGGGTGAGGAT | CUGUGGGGUGAGGAU |
| miR338 | AACUGCUGGACA | CCCCACAG | | | | | GTGTG (SEQ ID | GUGUG (SEQ ID |
| | CACUGAAGAAAC | (SEQ ID NO: | | | | | NO: 28) | NO: 49) |
| | U (SEQ ID | 7) | | | | | | |
| | NO: 1) | ACCCCACAGAAA | 35-54 | 58.9 | 55 | -4.1 | TCCAGCAGTTTCTGT | UCCAGCAGUUUCUGU |
| | | CTGCTGGA | | | | | GGGGT (SEQ ID | GGGGU (SEQ ID |
| | | (SEQ ID NO: | | | | | NO: 29) | NO: 50) |
| | | 8) | | | | | | |
| | | CAGAAACTGCTG | 40-59 | 55.1 | 50 | -3.55 | AGTGTGTCCAGCAGT | AGUGUGUCCAGCAGU |
| | | GACACACT | | | | | TTCTG (SEQ ID | UUCUG (SEQ ID |
| | | (SEQ ID NO: | | | | | NO: 30) | NO: 51) |
| | | 9) | | | | | | |
| miR424/ | UGAGCACACCAU | CCATTTGTGCTG | 1318-4337 | 56.8 | 50 | -3.14 | AACAGCAGCAGCACA | AACAGCAGCAGCACA |
| miR-15 | UUGUGCUGCUGC | CTGCTGTT | | | | | AATGG (SEQ ID | AAUGG (SEQ ID |
| | UGUUGUCGUGAA | (SEQ ID NO: | | | | | NO: 31) | NO: 52) |
| | AU (SEQ ID | 10) | | | | | | |
| | NO: 2) | TTGTGCTGCTGC | 1322-1341 | 58.9 | 55 | -3.61 | CGACAACAGCAGCAG | CGACAACAGCAGCAG |
| | | TGTTGTCG | | | | | CACAA (SEQ ID | CACAA (SEQ ID |
| | | (SEQ ID NO: | | | | | NO: 32) | NO: 32) |
| | | 11) | | | | | | |
| | | CTGCTGCTGTTG | 1327-1346 | 56.1 | 50 | -3.61 | TTTCACGACAACAGC | UUUCACGACAACAGC |
| | | TCGTGAAA | | | | | AGCAG (SEQ ID | AGCAG (SEQ ID |
| | | (SEQ ID NO: | | | | | NO: 33) | NO: 53) |
| | | 12) | | | | | | |
| miR218/ | GUUUGAAAGUAC | GAAAGUACUGAA | 1854-4873 | 49.3 | 40 | -6.84 | GTTTGTGCTTCAGTA | GUUUGUGCUUCAGUA |
| miR143 | UGAAGCACAAAC | GCACAAAC | | | | | CTTTC (SEQ ID | CUUUC (SEQ ID |
| | AUAUAUCAUCUC | (SEQ ID NO: | | | | | NO: 34) | NO: 54) |
| | U (SEQ ID | 13) | | | | | | |
| | NO: 3) | GCACAAACAUAU | 1866-1885 | 46.1 | 35 | -6.35 | GAGATGATATATGTT | GAGAUGAUAUAUGUU |
| | | AUCAUCUC | | | | | TGTGC (SEQ ID | UGUGC (SEQ ID |
| | | (SEQ ID NO: | | | | | NO: 35) | NO: 55) |
| | | 14) | | | | | | |
| | | AUCAUCUCUGUA | 1878-1897 | 48.9 | 40 | -3.65 | CAGAATGGTACAGAG | CAGAAUGGUACAGAG |
| | | CCAUUCUG | | | | | ATGAT (SEQ ID | AUGAU (SEQ ID |
| | | (SEQ ID NO: | | | | | NO: 36) | NO: 56) |
| | | 15) | | | | | | |
| miR- | GUCUGUCUUGAA | GUCUGUCUUGAA | 1805-1824 | 51 | 35 | | AAACAAGTTTCAAGA | AAACAAGUUUCAAGA |
| 301b/ | ACUUGUUUACCU | ACUUGUUU | | | | | CAGAC (SEQ ID | CAGAC (SEQ ID |
| 30c | UAAAAUUAUCAG | (SEQ ID NO: | | | | | NO: 37) | NO: 57) |
| | AA (SEQ ID | 16) | | | | | | |
| | NO: 4) | UCUUGAAACUUG | 1810-4829 | 49.1 | 30 | | AAGGTAAACAAGTTT | AAGGUAAACAAGUUU |
| | | UUUACCUU | | | | | CAAGA (SEQ ID | CAAGA (SEQ ID |
| | | (SEQ ID NO: | | | | | NO: 38) | NO: 58) |
| | | 17) | | | | | | |
| | | AAACUUGUUUAC | 1815-4834 | 45 | 20 | | ATTTTAAGGTAAACA | AUUUUAAGGUAAACA |
| | | CUUAAAAU | | | | | AGTTT (SEQ ID | AGUUU (SEQ ID |
| | | (SEQ ID NO: | | | | | NO: 39) | NO: 59) |
| | | 18) | | | | | | |
| | | UGUUUACCUUAA | 1820-4839 | 45 | 20 | | TGATAATTTTAAGGT | UGAUAAUUUUAAGGU |
| | | AAUUAUCA | | | | | AAACA (SEQ ID | AAACA (SEQ ID |
| | | (SEQ ID NO: | | | | | NO: 40) | NO: 60) |
| | | 19) | | | | | | |
| miR- | UUUGAAAUCUCC | UUUGAAAUCUCC | 1550-1569 | 45 | 55.3 | | GTGCAAGGGGAGATT | GUGCAAGGGGAGAUU |
| 148b | CCUUGCACUGAG | CCUUGCAC | | | | | TCAAA (SEQ ID | UCAAA (SEQ ID |
| | AUUAGUCGUCAG | (SEQ ID NO: | | | | | NO: 41) | NO: 61) |
| | A (SEQ ID | 20) | | | | | | |
| | NO: 5) | AAUCUCCCCUUG | 1555-1574 | 57.3 | 50 | | TCTCAGTGCAAGGGG | UCUCAGUGCAAGGGG |
| | | CACUGAGA | | | | | AGATT (SEQ ID | AGAUU (SEQ ID |
| | | (SEQ ID NO: | | | | | NO: 42) | NO: 62) |
| | | 21) | | | | | | |
| | | CCCCUUGCACUG | 1560-1579 | 57.3 | 50 | | ACTAATCTCAGTGCA | ACUAAUCUCAGUGCA |
| | | AGAUUAGU | | | | | AGGGG (SEQ ID | AGGGG (SEQ ID |
| | | (SEQ ID NO: | | | | | NO: 43) | NO: 63) |
| | | 22) | | | | | | |
| | | UGCACUGAGAUU | 1565-1584 | 55.3 | 45 | | TGACGACTAATCTCA | UGACGACUAAUCUCA |
| | | AGUCGUCA | | | | | GTGCA (SEQ ID | GUGCA (SEQ ID |
| | | (SEQ ID NO: | | | | | NO: 44) | NO: 64) |
| | | 23) | | | | | | |
| miR- | CCAAAGAAACAA | CCAAAGAAACAA | 5-24 | 512 | 40 | | GTGGATCTTTGTTTC | GUGGAUCUUUGUUUC |
| 3911 | AGAUCCACACAC | AGAUCCAC | | | | | TTTGG (SEQ ID | UUUGG (SEQ ID |
| | ACUCCUCACCCC | (SEQ ID NO: | | | | | NO: 45) | NO: 65) |
| | AC (SEQ ID | 24) | | | | | | |
| | NO: 6) | GAAACAAAGAUC | 10-29 | 512 | 40 | | TGTGTGTGGATCTTT | UGUGUGUGGAUCUUU |
| | | CACACACA | | | | | GTTTC (SEQ ID | GUUUC (SEQ ID |
| | | (SEQ ID NO: | | | | | NO: 46) | NO: 66) |
| | | 25) | | | | | | |
| | | AAAGAUCCACAC | 15-34 | 55.3 | 45 | | AGGAGTGTGTGTGGA | AGGAGUGUGUGUGGA |
| | | ACACUCCU | | | | | TCTTT (SEQ ID | UCUUU (SEQ ID |
| | | (SEQ ID NO: | | | | | NO: 47) | NO: 67) |
| | | 26) | | | | | | |
| | | UCCACACACACU | 20-39 | 61.4 | 60 | | GGGTGAGGAGTGTGT | GGGUGAGGAGUGUGU |
| | | CCUCACCC | | | | | GTGGA (SEQ ID | GUGGA (SEQ ID |
| | | (SEQ ID NO: | | | | | NO: 48) | NO: 68) |
| | | 27) | | | | | | |

### I. DEFINITIONS

Unless specific definitions are provided, the nomenclature used in connection with, and the procedures and techniques of, analytical chemistry, synthetic organic chemistry, and medicinal and pharmaceutical chemistry described herein are those well known and commonly used in the art. Unless otherwise indicated, the following terms have the following meanings.

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“Antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is a change in splicing of a pre-mRNA nucleic acid target. In certain embodiments, antisense activity is a change in the localization or structural formation of a nucleic acid target. In certain such embodiments, antisense activity is a decrease in foci formation of a nucleic acid target.

“Antisense compound” means a compound comprising an antisense oligonucleotide and optionally one or more additional features, such as a conjugate group or terminal group.

“Antisense oligonucleotide” means an oligonucleotide that (1) has a nucleobase sequence that is at least partially complementary to a target nucleic acid and that (2) is capable of producing an antisense activity in a cell or animal.

“Ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

“Bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

“Conjugate group” means a group of atoms that is directly or indirectly attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Duplex” means two oligomeric compounds that are paired. In certain embodiments, the two oligomeric compounds are paired via hybridization of complementary nucleobases.

“Fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage.

“Linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Non-bicyclic modified sugar” or “non-bicyclic modified sugar moiety” means a modified sugar moiety that comprises a modification, such as a substitutent, that does not form a bridge between two atoms of the sugar to form a second ring.

“Linked nucleosides” are nucleosides that are connected in a continuous sequence (i.e. no additional nucleosides are present between those that are linked).

“Mismatch” or “non-complementary” means a nucleobase of a first oligonucleotide that is not complementary with the corresponding nucleobase of a second oligonucleotide or target nucleic acid when the first and second oligomeric compound are aligned.

“MOE” means methoxyethyl. “2′-MOE” means a —OCH₂CH₂OCH₃ group at the 2′ position of a furanosyl ring.

“Motif” means the pattern of unmodified and/or modified sugar moieties, nucleobases, and/or internucleoside linkages, in an oligonucleotide.

“Naturally occurring” means found in nature.

“Nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase.

“Oligomeric compound” means a compound consisting of an oligonucleotide and optionally one or more additional features, such as a conjugate group or terminal group.

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“Pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

“Pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

“Phosphorus moiety” means a group of atoms comprising a phosphorus atom. In certain embodiments, a phosphorus moiety comprises a mono-, di-, or tri-phosphate, or phosphorothioate. In certain embodiments, a phosphorus moiety comprises a modified phosphate.

“Prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

“Single-stranded” in reference to an oligomeric compound means such a compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case it would no longer be single-stranded.

“Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Target nucleic acid” means a naturally occurring, identified nucleic acid. In certain embodiments, target nucleic acids are endogenous cellular nucleic acids, including, but not limited to RNA transcripts, pre-mRNA, mRNA, microRNA. In certain embodiments, target nucleic acids are viral nucleic acids. In certain embodiments, target nucleic acids are nucleic acids that an antisense compound is designed to affect.

“Target region” means a portion of a target nucleic acid to which an antisense compound is designed to hybridize.

“Terminal group” means a chemical group or group of atoms that is covalently linked to a terminus of an oligonucleotide.

“Unlocked nucleic acid” means a nucleoside comprising an unlocked sugar moiety. As used herein, “unlocked sugar” or “unlocked sugar moiety” means a modified sugar moiety comprising no rings. In certain embodiments, one of the bonds that is part of the ring of a furanosyl moiety is broken in an unlocked sugar.

### II. CERTAIN OLIGONUCLEOTIDES

In certain embodiments, the disclosure provides oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Certain Modified Nucleosides

Modified nucleosides comprise a modified sugar moiety or a modified nucleobase or both a modifed sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O-C₁-C₁₀ alkoxy, O-C₁-C₁₀ substituted alkoxy, O-C₁-C₁₀ alkyl, O-C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., U.S. Patent Publication 2013/0203836.

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N- substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂-N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379;Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191;; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al.,

US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

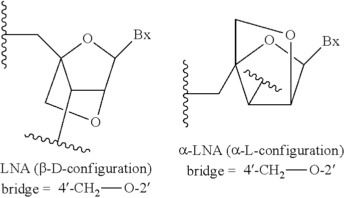

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

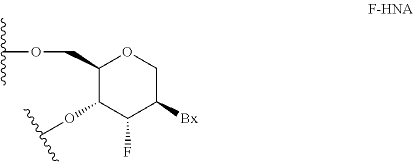

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

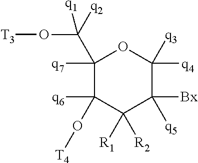

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁q₂, q₃, q₄, q₅, q₆ and q₇ are each H. In certain embodiments, at least one of q₁, q₂, q₃, q₄, q₅, q6 and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

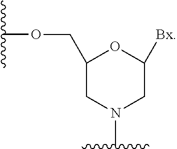

In certain embodiments, morpholinos may be modified, for example by adding or altering various substituent groups from the above morpholino structure. Such sugar surrogates are refered to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

B. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Motifs

In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

In certain embodiments, the present disclosure provides oligomeric compounds comprising one or more regions having a particular nucleoside motif. In certain embodiments, the 5′-terminal group and 5′-terminal nucleoside of an oligomeric compound of the present disclosure comprises a compound of Formula II, IIa, IIb, or IIc.

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification. In certain embodiments, modified oligonucleotides comprise or consist of a region having a fully modified sugar motif, wherein the fully modified sugar motif is an alternating motif. In certain such embodiments, the sugar moieties of the alternating motif alternate between 2′-OMe and 2′-F.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the oligonucleotide comprises a region of alternating internucleoside linkages. In certain such embodiments, the alternating internucleoside linkages are phosphorothioate and phosphate internucleoside linkages. In certain embodiments, the terminal internucleoside linkages are modified.

4. Certain Alternating Motifs

In certain embodiments, oligonucleotides of the present disclosure comprise one or more regions of alternating modifications. In certain embodiments, oligonucleotides comprise one or more regions of alternating nucleoside modifications. In certain embodiments, oligonucleotides comprise one or more regions of alternating linkage modifications. In certan embodiments, oligonucleotides comprise one or more regions of alternating nucleoside and linkage modifications.

In certain embodiments, oligonucleotides of the present disclosure comprise one or more regions of alternating 2′-F modified nucleosides and 2′-OMe modified nucleosides. In certain such embodiments, such regions of alternating 2′F modified and 2′OMe modified nucleosides also comprise alternating linkages. In certan such embodiments, the linkages at the 3′ position of the 2′-F modified nucleosides are phosphorothioate linkages. In certain such embodiments, the linkages at the 3′ position of the 2′OMe nucleosides are phosphate linkages.

In certain embodiments, oligomeric compounds comprise 2, 3, 4, 5, 6, 7, 8, 9, 10, or 11 regions of alternating modifications. Such regions may be contiguous or may be interupted by differently modified nucleosides or linkages.

In certan embodiments, alternating motifs have a pattern in which every other moiety is a first type or a second type, e.g., ABABA, etc. In certain embodiments, alternating motifs have a pattern in which the moieties of a first type and the moieties of a second type alternate in varied numbers. For example, oligonucleotide of the present disclosure may include one or more regions of any of the following alternating motifs:

AABBAA;

ABBABB;

AABAAB;

ABBABAABB;

ABABAA;

AABABAB;

ABABAA;

ABBAABBABABAA;

BABBAABBABABAA; or

ABABBAABBABABAA;

wherein A is a nucleoside or internucleoside linkage of a first type and B is a nucleoside or internucleoside linkage of a second type. In certain embodiments, A and B are each selected from 2′-F, 2′-OMe, BNA, DNA, and MOE.

In certain embodiments, compounds comprising such an alternating motif also comprise a 5′ terminal group and 5′-terminal nucleoside of formula II, IIa, IIb, or IIc.

5. Combinations of Motifs

It is to be understood, that certain of the above described motifs and modifications may be combined. Since a motif may comprise only a few nucleosides, a particular oligonucleotide may comprise two or more motifs. Oligonucleotides having any of the various nucleoside motifs described herein, may have any linkage motif. The lengths of the regions defined by a nucleoside motif and that of a linkage motif need not be the same. For example, non-limiting nucleoside motifs and sequence motifs are combined to show five examples in the table below. The first column of the table lists nucleosides and linkages by position from N1 (the 5′-end) to N20 (the 20^{th} position from the 5′-end). In certain embodiments, oligonucleotides of the present disclosure are longer than 20 nucleosides (the table is merely exemplary).

D. Certain Lengths

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

E. Certain Modified Oligonucleotides

In certain embodiments, the above modifications and motifs (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a fully modified sugar motif may be modified or unmodified and may or may not follow the modification pattern of the sugar modifications. Likewise, such fully modified sugar motifs may comprise one or more modified nucleobase independent of the pattern of the sugar modifications. Furthermore, in certain instances, an oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a fully modified sugar motif without further description may have any length, internucleoside linkage motif, and nucleobase motif. Unless otherwise indicated, all modifications are independent of nucleobase sequence.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### III. CERTAIN OLIGOMERIC COMPOUNDS

In certain embodiments, oligomeric compounds of the present disclosure are single-stranded. In certain embodiments, the disclosure provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphorus moieties (both unmodified phosphorus moieties and modified phosphorus moieties), protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. U.S.A., 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In certain embodiments, lipid conjugates include palmityl, C₂₂ alkyl, C₂₀ alkyl, C₁₆ alkyl, C₁₀ alkyl, and C₈ alkyl.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodimements, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### IV. CERTAIN ANTISENSE ACTIVITIES AND DISEASE STATES

In certain embodiments, the present disclosure provides antisense compounds that comprise or consist of an oligomeric compound comprising an antisense oliognucleotide, having a nucleobase sequence complementary to that of a target nucleic acid. In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity. In certain antisense activities, hybridization of an antisense compound to a target nucleic acid results in blocking of the action of an miRNA that targets the same site as the antisense compound.

In certain embodiments, the disclosure provides compounds and methods for antisense activity in a cell. In certain embodiments, the cell is in an animal. In certain embodiments, the animal is a human. In certain embodiments, the disclosure provides methods of administering a compound of the present disclosure to an animal to modulate one or more target nucleic acid. Antisense activities may be observed directly or indirectly. In certain embodiments, observation or detection of an antisense activity involves observation or detection of a change in an amount of a target nucleic acid or protein encoded by such target nucleic acid, and/or a phenotypic change in a cell or animal.

A. Epileptic Encephalopathy

Epileptic encephalopathies are characterized by epileptiform abnormalities associated with progressive cerebral dysfunction. Such related syndromes include early myoclonic encephalopathy and Ohtahara syndrome in the neonatal period, West syndrome and Dravet syndrome in infancy, myoclonic status in nonprogressive encephalopathies, and Lennox-Gastaut syndrome, Landau-Kleffner syndrome, and epilepsy with continuous spike waves during slow wave sleep in childhood and adolescences.

Current treatments are, for the most part, not significantly effective. Dietary therapy has been tried with some success, usch a ketogenic diet. Neurosurgery is sometimes indicated in select cases of cerebral malformations. The prognosis is uniformly poor with survivors left with severe psychomotor retardation.

B. Neurodevelopmental Disorders

Neurodevelopmental disorder is a mental disorder. A narrower use of the term refers to a disorder of brain function which affects emotion, learning ability, self-control and memory and which unfolds as the individual grows. Neurodevelopmental disorders tend to last for a person's entire lifetime. Disorders currently considered neurodevelopmental are definetely of one of these types:
Intellectual disability (ID) or intellectual and developmental disability (IDD), previously called mental retardation
Autism spectrum disorders, such as Asperger's syndrome or Kanner syndrome
Motor disorders including developmental coordination disorder and stereotypic movement disorder
Tic disorders including Tourette's syndrome
Traumatic brain injury (including congenital injuries such as those that cause cerebral palsy)
Communication, speech and language disorders
Genetic disorders, such as fragile-X syndrome, Down syndrome, attention deficit hyperactivity disorder, schizophrenia, schizotypal disorder, hypogonadotropic hypogonadal syndromes
Disorders due to neurotoxicants like fetal alcohol spectrum disorder, Minamata disease caused by mercury, behavioral disorders including conduct disorder etc. caused by other heavy metals, such as lead, chromium, platinum, etc., hydrocarbons like dioxin, PBDEs and PCBs, medications and illegal drugs, like cocaine and others.

The development of the nervous system including the brain is orchestrated, tightly regulated, and genetically encoded process with clear influence from the environment. This suggests that any deviation from this program early in life can result in neurodevelopmental disorders and, depending on specific timing, might lead to distinct pathology later in life. Because of that, there are many causes of neurodevelopmental disorder, which can range from deprivation, genetic and metabolic diseases, immune disorders, infectious diseases, nutritional factors, physical trauma, and toxic and environmental factors.

Some neurodevelopmental disorders—such as autism and other pervasive developmental disorders—are considered multifactorial syndromes with many causes but more specific neurodevelopmental manifestation.

Deprivation from social and emotional care causes severe delays in brain and cognitive development. Studies with children growing up in Romanian orphanages during Nicolae Ceau [symbol] escu's regime reveal profound effects of social deprivation and language deprivation on the developing brain. These effects are time dependent. The longer children stayed in negligent institutional care, the greater the consequences. By contrast, adoption at an early age mitigated some of the effects of earlier institutionalization (abnormal psychology).

A prominent example of a genetically determined neurodevelopmental disorder is Trisomy 21, also known as Down syndrome. This disorder usually results from an extra chromosome 21, although in uncommon instances it is related to other chromosomal abnormalities such as translocation of the genetic material. It is characterized by short stature, epicanthal (eyelid) folds, abnormal fingerprints, and palm prints, heart defects, poor muscle tone (delay of neurological development) and mental retardation (delay of intellectual development).

Less commonly known genetically determined neurodevelopmental disorders include Fragile X syndrome. Fragile X syndrome was first described in 1943 upon the studying persons with family history of sex-linked “mental defects”. Rett syndrome, another X-linked disorder, produces severe functional limitations. Williams syndrome is caused by small deletions of genetic material from chromosome 7. The most common recurrent Copy Number Variannt disorder is 22q11.2 deletion syndrome (formerly DiGeorge or velocardiofacial syndrome), followed by Prader-Willi syndrome and Angelman syndrome.

Immune reactions during pregnancy, both maternal and of the developing child, may produce neurodevelopmental disorders. One typical immune reaction in infants and children is PANDAS, or Pediatric Autoimmune Neuropsychiatric Disorders Associated with Streptococcal infection. Another disorder is Sydenham's chorea, which results in more abnormal movements of the body and fewer psychological sequellae. Both are immune reactions against brain tissue that follow infection by Streptococcus bacteria. Susceptibility to these immune diseases may be genetically determined, so sometimes several family members may suffer from one or both of them following an epidemic of Strep infection.

Systemic infections can result in neurodevelopmental consequences, when they occur in infancy and childhood of humans, but would not be called a primary neurodevelopmental disorder per se, as for example HIV Infections of the head and brain, like brain abscesses, meningitis or encephalitis have a high risk of causing neurodevelopmental problems and eventually a disorder. For example, measles can progress to subacute sclerosing panencephalitis.

A number of infectious diseases can be transmitted congenitally (either before or at birth), and can cause serious neurodevelopmental problems, as for example the viruses HSV, CMV, rubella (congenital rubella syndrome), Zika virus, or bacteria like Treponema pallidum in congenital syphilis, which may progress to neurosyphilis if it remains untreated. Protozoa like Plasmodium or Toxoplasma which can cause congenital toxoplasmosis with multiple cysts in the brain and other organs, leading to a variety of neurological deficits.

Metabolic disorders in either the mother or the child can cause neurodevelopmental disorders. Two examples are diabetes mellitus (a multifactorial disorder) and phenylketonuria (an inborn error of metabolism). Many such inherited diseases may directly affect the child's metabolism and neural development but less commonly they can indirectly affect the child during gestation.

In a child, type 1 diabetes can produce neurodevelopmental damage by the effects of excess or insufficient glucose. The problems continue and may worsen throughout childhood if the diabetes is not well controlled. Type 2 diabetes may be preceded in its onset by impaired cognitive functioning.

A non-diabetic fetus can also be subjected to glucose effects if its mother has undetected gestational diabetes. Maternal diabetes causes excessive birth size, making it harder for the infant to pass through the birth canal without injury or it can directly produce early neurodevelopmental deficits. Usually the neurodevelopmental symptoms will decrease in later childhood.

Phenylketonuria, also known as PKU, can induce neurodevelopmental problems and children with PKU require a strict diet to prevent mental retardation and other disorders. In the maternal form of PKU, excessive maternal phenylalanine can be absorbed by the fetus even if the fetus has not inherited the disease. This can produce mental retardation and other disorders.

Nutrition disorders and nutritional deficits may cause neurodevelopmental disorders, such as spina bifida, and the rarely occurring anencephaly, both of which are neural tube defects with malformation and dysfunction of the nervous system and its supporting structures, leading to serious physical disability and emotional sequelae. The most common nutritional cause of neural tube defects is folic acid deficiency in the mother, a B vitamin usually found in fruits, vegetables, whole grains, and milk products. (Neural tube defects are also caused by medications and other environmental causes, many of which interfere with folate metabolism, thus they are considered to have multifactorial causes.) Another deficiency, iodine deficiency, produces a spectrum of neurodevelopmental disorders ranging from mild emotional disturbance to severe mental retardation.

Excesses in both maternal and infant diets may cause disorders as well, with foods or food supplements proving toxic in large amounts. For example, it has been discovered that iron supplementation in baby formula can be linked to lowered I.Q. and other neurodevelopmental delays.

Brain trauma in the developing human is a common cause (over 400,000 injuries per year in the U.S. alone, without clear information as to how many produce developmental sequellae) of neurodevelopmental syndromes. It may be subdivided into two major categories, congenital injury (including injury resulting from otherwise uncomplicated premature birth) and injury occurring in infancy or childhood. Common causes of congenital injury are asphyxia (obstruction of the trachea), hypoxia (lack of oxygen to the brain) and the mechanical trauma of the birth process itself.

Traditionally, genetic abnormalities in neurodevelopmental disorders were detected using karyotype analysis, which found 5% of relevant disorders. As of 2017, chromosomal microarray analysis (CMA) has replaced karyotyping, because of its greater diagnostic yield in about 20% of cases, detecting smaller chromosome abnormalities. It is the first line genomic test.

New descriptions include the term Copy-number variants (CNVs), which are losses or gains of chromosomal regions greater than 1 kb in length. CNVs are mentioned with the chromosomal band(s) they involve and their genome sequence coordinates. CNVs can be nonrecurrent and recurrent.

With CMA, costs of testing have increased from $800 US to $1500. Guidelines from the American College of Medical Genetics and Genomics and the American Academy of Pediatrics recommend CMA as standard of care in the US.

### V. TARGET NUCLEIC ACIDS

In certain embodiments, antisense compounds comprise or consist of an oligonucleotide comprising a region that is complementary to an miRNA binding site in a target nucleic acid. In certain embodiments, the target nucleic acid is an endogenous RNA molecule, and in particular a miRNA target site in an mRNA molecule. In certain embodiments, the target nucleic acid is a mRNA for STXBP1, SCN1A, SCN2A, SCN8A, SLC6A1, or MECP2.

In certain embodiments, antisense compounds comprise antisense oligonucleotides that are complementary to the target nucleic acid over the entire length of the oligonucleotide. In certain embodiments, such oligonucleotides are 99% complementary to a mRNA transcript. In certain embodiments, such oligonucleotides are 95% complementary to a mRNA transcript. In certain embodiments, such oligonucleotides are 90% complementary to a mRNA transcript. In certain embodiments, such oligonucleotides are 85% complementary to a mRNA transcript. In certain embodiments, such oligonucleotides are 80% complementary to a mRNA transcript. In certain embodiments, antisense oligonucleotides are at least 80% complementary to a mRNA transcript over the entire length of the oligonucleotide and comprise a region that is 100% or fully complementary to a mRNA transcript. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, the oligonucleotides of antisense compounds comprise one or more mismatched nucleobases relative to the target nucleic acid, e.g., a mRNA transcript. In certain such embodiments, antisense activity against the target is reduced by such mismatch, but activity against a non-target is reduced by a greater amount. Thus, in certain such embodiments selectivity of the antisense compound is improved. In certain embodiments, antisense activity against the target is increased by such mismatch. In certain such embodiments, the position or positions of the mismatch affect the antisense activity of the compound. In certain such embodiments, the mismatch is at position 21, 20, 19, 18, 17, 16, 15, 14, 13, 12, 11, 10, 9, 8, 7, 6, 5, 4, 3, 2, and/or 1 from the 5′-end of the oligonucleotide. In certain embodiments, the oligonucleotides of antisense compounds comprise two or more mismatches relative to the target nucleic acid.

### VI. Certain Pharmaceutical Compositions

In certain embodiments, the present disclosure provides pharmaceutical compositions comprising one or more antisense compound, oligomeric compound, and/or a salt thereof. In certain such embodiments, the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present disclosure to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for intrathecal administration. In certain embodiments, a pharmaceutical composition is prepared for administration by injection (e.g., intravenous, subcutaneous, intramuscular, intrathecal, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

Although the sequence listing accompanying this filing identifies each sequence as either “RNA” or “DNA” as required, in reality, those sequences may be modified with any combination of chemical modifications. One of skill in the art will readily appreciate that such designation as RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Included in the compounds provided herein are all such possible isomers, including their racemic and optically pure forms, unless specified otherwise. Likewise, all cis- and trans-isomers, E and Z isomers, and tautomeric forms are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### VII. EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular modification appears at a particular position, other modifications at the same position are considered suitable, unless otherwise indicated.

### Example 1—Synthesis of Oligomeric Compounds

The oligonucleotides and oligomeric compounds provided herein can be prepared by any of the applicable techniques of organic synthesis. Many such techniques are well known in the art and described in Compendium of Organic Synthetic Methods, John Wiley & Sons, New York: Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade Jr., 1980; Vol. 5, Leroy G. Wade Jr., 1984; and Vol. 6, Michael B. Smith; as well as March, J., Advanced Organic Chemistry, 3rd Edition, John Wiley & Sons, New York, 1985; Comprehensive Organic Synthesis. Selectivity, Strategy & Efficiency in Modern Organic Chemistry, in 9 Volumes, Barry M. Trost, Editor-in-Chief, Pergamon Press, New York, 1993; Advanced Organic Chemistry, Part B: Reactions and Synthesis, 4th Edition; Carey and Sundberg, Kluwer Academic/Plenum Publishers, New York, 2001; Advanced Organic Chemistry, Reactions, Mechanisms, and Structure, 2nd Edition, March, McGraw Hill, 1977; Greene, T. W., and Wutz, P. G. M., Protecting Groups in Organic Synthesis, 4th Edition, John Wiley & Sons, New York, 1991; and Larock, R. C., Comprehensive Organic Transformations, 2nd Edition, John Wiley & Sons, New York, 1999. Protocols for Oligonucleotides and Analogs, Agrawal, Ed., Humana Press, 1993, and/or RNA: Scaringe, Methods, 2001, 23, 206-217; Gait et al., Applications of Chemically synthesized RNA in RNA:Protein Interactions, Smith, Ed., 1998, 1-36; Gallo et al., Tetrahedron , 2001, 57, 5707-5713. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. No. 4,725,677 and U.S. Re. 34,069. Additional methods applicable to synthesis of the compounds herein may be found in U.S. Pat. 8,993,738 and U.S. Patent Publication 2014/0316121.

### Example 2

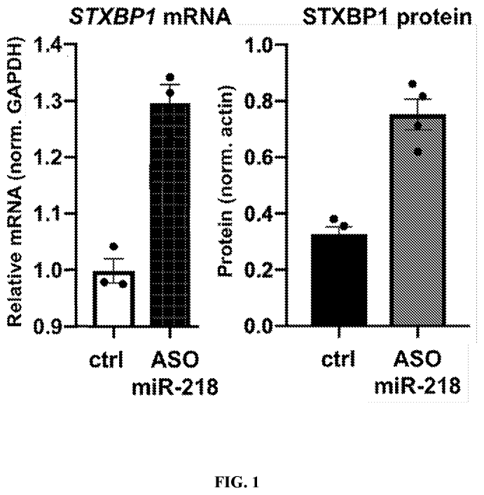

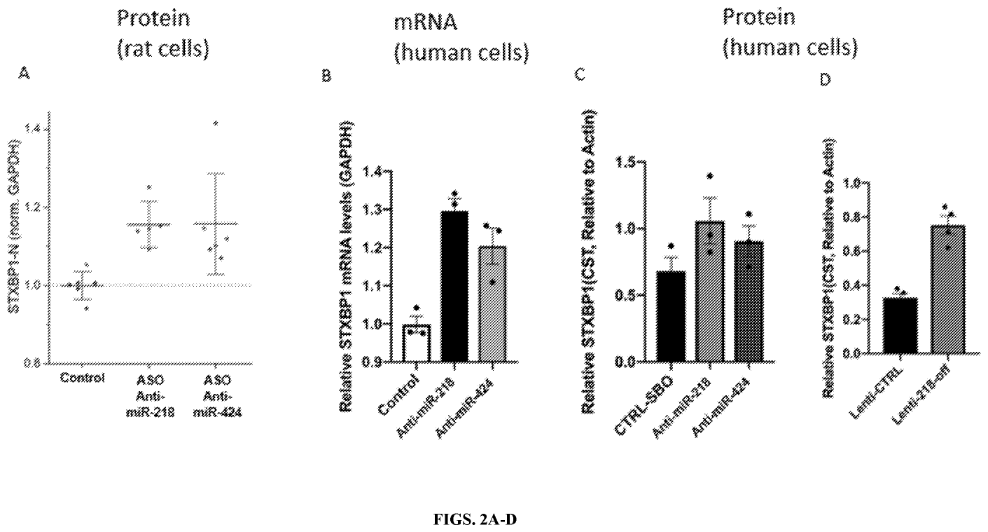

STXBP1 mRNA and protein levels were analyzed in a neuronal cell line (SH-SY5Y) transfected with antisense oligonucleotides to inhibit miR-218. (FIG. 1). Disrupting miR-218 relieved the inhibition of STXBP1, resulting in a statistically significant ˜2-fold increase in protein expression.

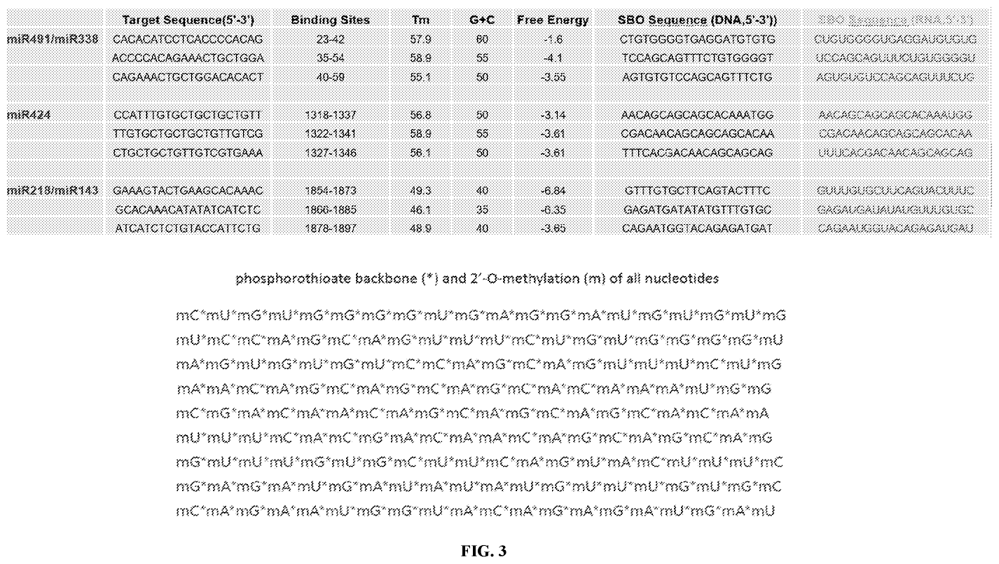

Elevated STXBP1 protein levels were detected via western blot 48 h after transfection of rat glioblastoma line (PC12 cells) with an ASO to inhibit miR-218 and miR-424 (FIG. 2A). Increased mRNA levels of STXBP1 were detected by qPCR (FIG. 2B) and increased protein levels of STXBP1 were detected by western blot (FIG. 2C) 48 h after transfection of human neuroblastoma line (SH-SY5Y cells). Increased STXBP1 protein levels were detected via western blot 48 h following lentiviral transduction of ASO against miR-218 in SH-SY5Y cells (FIG. 2D).

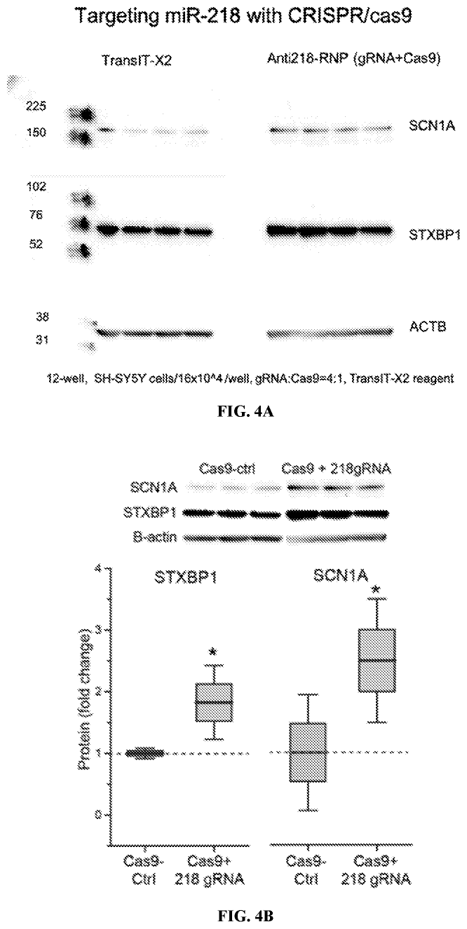

Inhibition of miR-218 via Cas9-mediated excision upregulated expression of STXBP1 and SCN1A, both miR-218 targets, in human neuronal cells (FIGS. 4A&B).

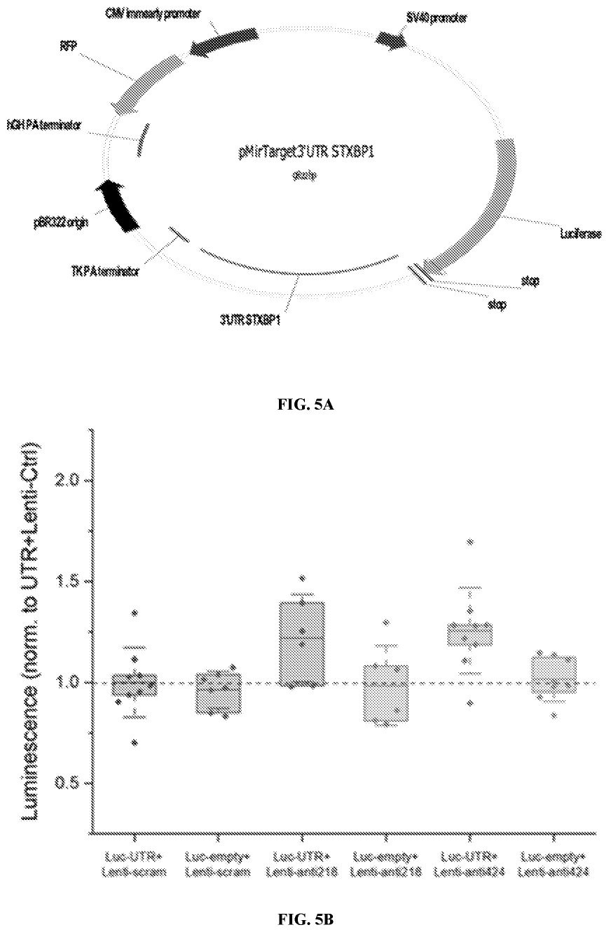

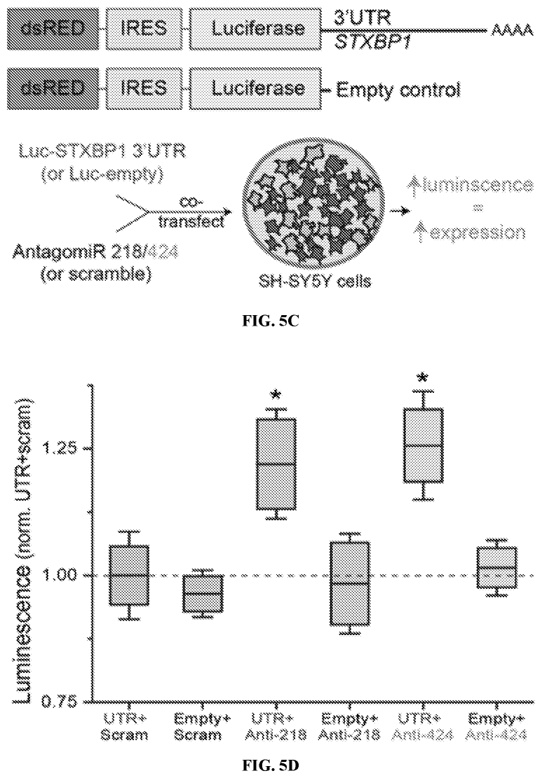

A luciferase reporter was generated to screen ASOs for the ability to upregulate STXBP1 expression. For this, the 3′UTR of STXBP1 was cloned downstream of a luciferase reporter gene. If an ASO disrupts miR-mediated repression of STXBP1 by binding to its 3′UTR, this results in increased light emitted from the sample (FIGS. 5A&C). Reporter construct transfected into SH-SY5Y cells revealed increased STXBP1 3′UTR expression upon inhibition of miR-218 and miR-424 (FIGS. 5B&D).

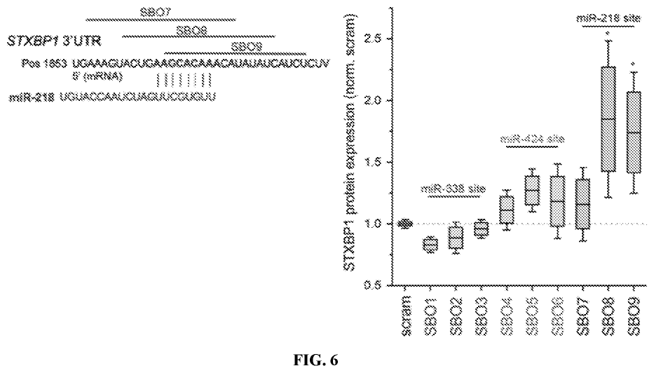

The left panel of FIG. 6 provides a schematic depicting miR-218 (SEQ ID NO: 69) binding site in the 3′UTR of STXBP1 (nucleotides 4-37 of SEQ ID NO: 3), and target sites of SBOs 7, 8, and 9. A similar 3 SBO strategy was used to flank the miR-424 and 338 sites on STXBP1 which are toward the 5′ end of the 3′ UTR. The data in the right panel of FIG. 6 provide results of triplicate experiments treating SH-SY5Y cells with 10 pmol/well of the 9 SBOs that target the three putative miR-binding sites.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed:

1. A compound comprising a single-stranded oligonucleotide, wherein the single-stranded oligonucleotide consists of 13 to 30 linked nucleosides and has a nucleobase sequence comprising a complementary region having at least 7 contiguous nucleobases complementary to an equal-length portion within a target region of a target nucleic acid, wherein the target nucleic acid is located in an mRNA transcript selected from the group consisting of STXBP1, SCN1A, SCN2A, SCN8A, SLC6A1, and MECP2.

2. The compound of claim 1, wherein the target is an STXBP1 mRNA.

3. The compound of claim 1, wherein the target is an SCN1A mRNA.

4. The compound of claim 1, wherein the target is an SCN2A mRNA.

5. The compound of claim 1, wherein the target is an SCN8A mRNA.

6. The compound of claim 1, wherein the target is an SLC6A1 mRNA.

7. The compound of claim 1, wherein the target is a MECP2 mRNA.

8. The compound of any of claims 1 to 4, wherein the complementary region comprises at least 10, 12, 14, 16, 18, or 20 contiguous nucleobases complementary to an equal-length portion within the target region of the mRNA transcript.

9. The compound of claim 2, wherein the target region at least partially overlaps a binding site for miR218, miR148b, miR30b, miR30c, miR424, miR15b, miR3911, miR338, or miR942.

10. The compound of claim 2, wherein the target region comprises an at least 10, 12, 14, 16, 18, or 20 continguous nucleotide segment of any one of:
(SEQ ID NO: 1)
UCACCCCACAGAAACUGCUGGACACACUGAAGAAACU,
(SEQ ID NO: 2)
UGAGCACACCAUUUGUGCUGCUGCUGUUGUCGUGAAAU,
(SEQ ID NO: 3)
GUUUGAAAGUACUGAAGCACAAACAUAUAUCAUCUCU,
(SEQ ID NO: 4)
GUCUGUCUUGAAACUUGUUUACCUUAAAAUUAUCAGAA,
(SEQ ID NO: 5)
UUUGAAAUCUCCCCUUGCACUGAGAUUAGUCGUCAGA,
or
(SEQ ID NO: 6)
CCAAAGAAACAAAGAUCCACACACACUCCUCACCCCAC.

11. The compound of claim 2, wherein the target region is a sequence selected from
(SEQ ID NO: 7)
CACACATCCTCACCCCACAG,
(SEQ ID NO: 8)
ACCCCACAGAAACTGCTGGA,
(SEQ ID NO: 9)
CAGAAACTGCTGGACACACT,
(SEQ ID NO: 10)
CCATTTGTGCTGCTGCTGTT,
(SEQ ID NO: 11)
TTGTGCTGCTGCTGTTGTCG,
(SEQ ID NO: 12)
CTGCTGCTGTTGTCGTGAAA,
(SEQ ID NO: 13)
GAAAGTACTGAAGCACAAAC,
(SEQ ID NO: 14)
GCACAAACATATATCATCTC,
(SEQ ID NO: 15)
ATCATCTCTGTACCATTCTG,
(SEQ ID NO: 16)
GUCUGUCUUGAAACUUGUUU,
(SEQ ID NO: 17)
UCUUGAAACUUGUUUACCUU,
(SEQ ID NO: 18)
AAACUUGUUUACCUUAAAAU,
(SEQ ID NO: 19)
UGUUUACCUUAAAAUUAUCA,
(SEQ ID NO: 20)
UUUGAAAUCUCCCCUUGCAC,
(SEQ ID NO: 21)
AAUCUCCCCUUGCACUGAGA,
(SEQ ID NO: 22)
CCCCUUGCACUGAGAUUAGU,
(SEQ ID NO: 23)
UGCACUGAGAUUAGUCGUCA,
(SEQ ID NO: 24)
CCAAAGAAACAAAGAUCCAC,
(SEQ ID NO: 25)
GAAACAAAGAUCCACACACA,
(SEQ ID NO: 26)
AAAGAUCCACACACACUCCU,
or
(SEQ ID NO: 27)
UCCACACACACUCCUCACCC.

12. The compound of claim 2, wherein the compound nucleobase sequence comprises a sequence that is at least 70% identical to a complement of an at least 10, 12, 14, 16, 18, or 20 continguous nucleotide segment of any one of:
(SEQ ID NO: 1)
UCACCCCACAGAAACUGCUGGACACACUGAAGAAACU,
(SEQ ID NO: 2)
UGAGCACACCAUUUGUGCUGCUGCUGUUGUCGUGAAAU,
(SEQ ID NO: 3)
GUUUGAAAGUACUGAAGCACAAACAUAUAUCAUCUCU,
(SEQ ID NO: 4)
GUCUGUCUUGAAACUUGUUUACCUUAAAAUUAUCAGAA,
(SEQ ID NO: 5)
UUUGAAAUCUCCCCUUGCACUGAGAUUAGUCGUCAGA,
or
(SEQ ID NO: 6)
CCAAAGAAACAAAGAUCCACACACACUCCUCACCCCAC.

13. The compound of claim 2, wherein (a) the compound DNA sequence is
(SEQ ID NO: 28)
CTGTGGGGTGAGGATGTGTG,
(SEQ ID NO: 29)
TCCAGCAGTTTCTGTGGGGT,
(SEQ ID NO: 30)
AGTGTGTCCAGCAGTTTCTG,
(SEQ ID NO: 31)
AACAGCAGCAGCACAAATGG,
(SEQ ID NO: 32)
CGACAACAGCAGCAGCACAA,
(SEQ ID NO: 33)
TTTCACGACAACAGCAGCAG,
(SEQ ID NO: 34)
GTTTGTGCTTCAGTACTTTC,
(SEQ ID NO: 35)
GAGATGATATATGTTTGTGC,
(SEQ ID NO: 36)
CAGAATGGTACAGAGATGAT,
(SEQ ID NO: 37)
AAACAAGTTTCAAGACAGAC,
(SEQ ID NO: 38)
AAGGTAAACAAGTTTCAAGA,
(SEQ ID NO: 39)
ATTTTAAGGTAAACAAGTTT,
(SEQ ID NO: 40)
TGATAATTTTAAGGTAAACA,
(SEQ ID NO: 41)
GTGCAAGGGGAGATTTCAAA,
(SEQ ID NO: 42)
TCTCAGTGCAAGGGGAGATT,
(SEQ ID NO: 43)
ACTAATCTCAGTGCAAGGGG,
(SEQ ID NO: 44)
TGACGACTAATCTCAGTGCA,
(SEQ ID NO: 45)
GTGGATCTTTGTTTCTTTGG,
(SEQ ID NO: 46)
TGTGTGTGGATCTTTGTTTC,
(SEQ ID NO: 47)
AGGAGTGTGTGTGGATCTTT,
or
(SEQ ID NO: 48)
GGGTGAGGAGTGTGTGTGGA;
and/or
(b) the compound RNA sequence is
(SEQ ID NO: 49)
CUGUGGGGUGAGGAUGUGUG,
(SEQ ID NO: 50)
UCCAGCAGUUUCUGUGGGGU,
(SEQ ID NO: 51)
AGUGUGUCCAGCAGUUUCUG,
(SEQ ID NO: 52)
AACAGCAGCAGCACAAAUGG,
(SEQ ID NO: 32)
CGACAACAGCAGCAGCACAA,
(SEQ ID NO: 53)
UUUCACGACAACAGCAGCAG,
(SEQ ID NO: 54)
GUUUGUGCUUCAGUACUUUC,
(SEQ ID NO: 55)
GAGAUGAUAUAUGUUUGUGC,
(SEQ ID NO: 56)
CAGAAUGGUACAGAGAUGAU,
(SEQ ID NO: 57)
AAACAAGUUUCAAGACAGAC,
(SEQ ID NO: 58)
AAGGUAAACAAGUUUCAAGA,
(SEQ ID NO: 59)
AUUUUAAGGUAAACAAGUUU,
(SEQ ID NO: 60)
UGAUAAUUUUAAGGUAAACA,
(SEQ ID NO: 61)
GUGCAAGGGGAGAUUUCAAA,
(SEQ ID NO: 62)
UCUCAGUGCAAGGGGAGAUU,
(SEQ ID NO: 63)
ACUAAUCUCAGUGCAAGGGG,
(SEQ ID NO: 64)
UGACGACUAAUCUCAGUGCA,
(SEQ ID NO: 65)
GUGGAUCUUUGUUUCUUUGG,
(SEQ ID NO: 66)
UGUGUGUGGAUCUUUGUUUC,
(SEQ ID NO: 67)
AGGAGUGUGUGUGGAUCUUU,
or
(SEQ ID NO: 68)
GGGUGAGGAGUGUGUGUGGA.

14. The compound of any of claims 1 to 13, wherein the single-stranded oligonucleotide comprises a a terminal group at the 5′-end of the oligonucleotide and the 5′-terminal nucleoside and terminal group of the compound has Formula I: 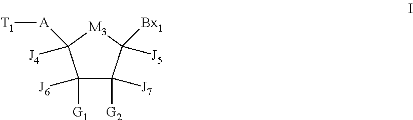
wherein:
T₁ is a phosphorus moiety;
A has a formula selected from among: 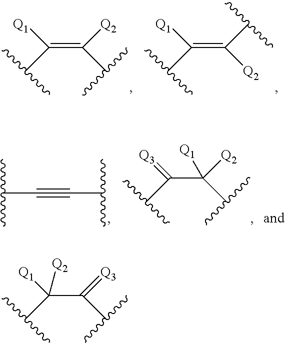
Q₁ and Q₂ are each independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl, and N(R₃)(R₄);
Q₃ is selected from among: O, S, N(R₅), and C(R₆)(R₇);
each R₃, R₄ R₅, R₆ and R₇ is independently selected from among: H, C₁-C₆ alkyl, substituted C₁-C₆ alkyl, and C₁-C₆ alkoxy;
M₃ is selected from among: O, S, NR₁₄, C(R₁₅)(R₁₆), C(R₁₅)(R₁₆)C(R₁₇)(R₁₈), C(R₁₅)═C(R₁₇), OC(R₁₅)(R₁₆), and OC(R₁₅)(Bx₂);
R₁₄ is selected from among: H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl;
R₁₅, R₁₆, R₁₇ and Rig are each independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl;
if Bx₂ is present, then Bx₂ is a nucleobase and Bx₁ is selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl;
if Bx₂ is not present, then Bx₁ is a nucleobase;
either each of J₄, J₅, J₆ and J₇ is independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl;
or J₄ forms a bridge with one of J₅ or J₇ wherein the bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are independently selected from among: H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, and substituted C₂-C₆ alkynyl;
each R₁₉, R₂₀ and R₂₁ is independently selected from among: H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
one of G₁ and G₂ is selected from among: H, OH, halogen and O-[C(R₈)(R₉)]ₙ—[(C═O)ₘ—X₁]₁—Z; and the other of G₁ and G₂ is: O-T₂;
T₂ is an internucleoside linking group linking the 5′-terminal nucleoside of Formula I to the remainder of the oligonucleotide;
each R₈ and R₉ is independently selected from among: H, halogen, C₁-C₆ alkyl, and substituted C₁-C₆ alkyl;
X₁ is O, S or N(E₁);
Z is selected from among: H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, and N(E₂)(E₃);
E₁, E₂ and E₃ are each independently selected from among: H, C₁-C₆ alkyl, and substituted C₁-C₆ alkyl;
n is from 1 to 6;
m is 0 or 1;
j is 0 or 1;
provided that, if j is 1, then Z is other than halogen or N(E₂)(E₃);
each substituted group comprises one or more optionally protected substituent groups independently selected from among: a halogen, OJ₁, N(J₁)(J₂), =NJ₁, SJ₁, N₃, CN, OC(═X₂)J₁, OC(═X₂)N(J₁)(J₂), and C(═X₂)N(J₁)(J₂);
X₂ is O, S or NJ₃; and
each J₁, J₂ and J₃ is independently selected from among: H and C₁-C₆ alkyl.

15. The compound of claim 14, wherein M₃ is selected from among: O, CH═CH, OCH₂, and OC(H)(Bx₂).

16. The compound of claim 14, wherein M₃ is 0.

17. The compound of any of claims 14 to 16, wherein each of J₄, J₅, J₆ and J₇ is H.

18. The compound of any of claims 14 to 17, wherein J₄ forms a bridge with either J₅ or J₇.

19. The compound of any of claims 14 to 18, wherein A has the formula: 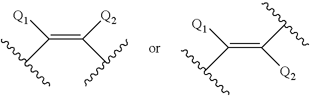
wherein Q₁ and Q₂ are each independently selected from among: H, halogen, C₁-C₆ alkyl, substituted C₁-C₆ alkyl, C₁-C₆ alkoxy, and substituted C₁-C₆ alkoxy.

20. The compound of claim 19, wherein each of Q₁ and Q₂ is H.

21. The compound of claim 19, wherein Q₁ and Q₂ are each independently selected from among: H and a halogen.

22. The compound of claim 19, wherein one of Q₁ and Q₂ is H and the other of Q₁ and Q₂ is F, CH₃ or OCH₃.

23. The compound of any of claims 14 to 22, wherein T₁ has the formula: 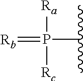
wherein:
Rₐ and R_{c} are each independently selected from among: hydroxyl, protected hydroxyl, thiol, protected thiol, C₁-C₆ alkyl, substituted C₁-C₆ alkyl, C₁-C₆ alkoxy, substituted C₁-C₆ alkoxy, amino, protected amino or substituted amino; and
R_{b} is O or S.

24. The compound of claim 23, wherein R_{b} is O and Rₐ and R_{b} are each, independently selected from among: OH, OCH₃, OCH₂CH₃, OCH(CH₃)₂.

25. The compound of any of claims 14 to 24, wherein one of G₁ and G₂ is selected from among: a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁₀)(R₁₁), O(CH₂)₂—ON(R₁₀)(R₁₁), O(CH₂)₂—O(CH₂)₂—N(R₁₀)(R₁₁), OCH₂C(═O)—N(R₁₀)(R₁₁), OCH₂C(═O)—N(R₁₂)—(CH₂)₂—N(R₁₀)(R₁₁), and O(CH₂)₂—N(R₁₂)—C(═NR₁₃)[N(R₁₀)(R₁₁)]; wherein R₁₀, R₁₁, R₁₂ and R₁₃ are each, independently, H or C₁-C₆ alkyl.

26. The compound of any of claims 14 to 25, wherein one of G₁ and G₂ is selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.

27. The compound of any of claims 14 to 26, wherein one of G₁ and G₂ is selected from among: F, OCH₃, and O(CH₂)₂—OCH₃.

28. The compound of claim 27, wherein one of G₁ and G₂ is O(CH₂)₂—OCH₃.

29. The compound of any of claims 14 to 28, wherein the 5′-terminal nucleoside and terminal group of the compound has Formula III:

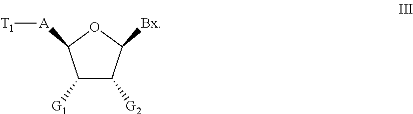

30. The compound of claim 29, wherein A has the formula: 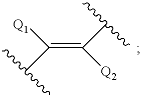
wherein Q₁ and Q₂ are each independently selected from among: H, a halogen, C₁-C₆ alkyl, substituted C₁-C₆ alkyl, C₁-C₆ alkoxy, and substituted C₁-C₆ alkoxy.

31. The compound of claim 30, wherein Q₁ and Q₂ are each independently selected from among: H, F, CH₃, and OCH₃.

32. The compound of any of claims 14 to 31, wherein the 5′-terminal nucleoside and the terminal group has Formula V: 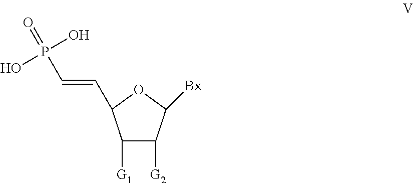
wherein:
Bx is selected from among: uracil, thymine, cytosine, 5-methyl cytosine, adenine, and guanine;
one of G₁ and G₂ is selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂-N(CH₃)₂, OCH₂C(═O)—N(H)CH₃, OCH₂C(═O)—N(H)—(CH₂)₂—N(CH₃)₂ and OCH₂—N(H)—C(═NH)NH₂;
and the other of G₁ and G₂ is O-T₂, wherein T₂ is a phosphorothioate internucleoside linking group linking the compound of Formula V to the remainder of the oligonucleotide.

33. The compound of any of claims 1 to 32, wherein the single-stranded oligonucleotide comprises at least two modified sugar moieties.

34. The compound of any of claims 33, wherein each modified sugar moiety is independently selected from among: 2′-F, 2′-MOE, 2′-OMe, LNA, F-HNA, and cEt.

35. The compound of claim 34, wherein the modified oligonucleotide is fully modified, such as with 2′-OMe, optionally further comprising a phosphorothioate backbone.

36. The compound of any of claims 33 to 35, wherein the modified oligonucleotide comprises at least one region having sugar motif:
-[(A)ₓ-(B)_{y}-(A)_{z}]_{q}-
wherein
A is a modified nucleoside of a first type,
B is a modified nucleoside of a second type;
each x and each y is independently 1 or 2;
z is 0 or 1;
q is 3-15.

37. The compound of claim 36, wherein each x and each y is 1.

38. The compound of claim 36 or 37, wherein A is a modified nucleoside selected from among: a 2′-F, a 2′-OMe, and a F-HNA modified nucleoside.

39. The compound of claim 36 or 37, wherein B is a modified nucleoside selected from among: a 2′-F, a 2′-OMe, and a F-HNA modified nucleoside.

40. The compound of any of claims 36 to 39, wherein A is a 2′-F modified nucleoside and B is a 2′-OMe modified nucleoside.

41. The compound of any of claims 36 to 39, wherein B is a 2′-F modified nucleoside and A is a 2′-OMe modified nucleoside.

42. The compound of any of claims 33 to 41, wherein the modified oligonucleotide comprises 1-4 3′-terminal nucleosides, each comprising the same modified sugar moiety, wherein the modified sugar moiety of the 1-4 3′-terminal nucleosides is different from the modified sugar moiety of the immediately adjacent nucleoside.

43. The compound of claim 42, wherein the 3′-terminal nucleosides are each 2′-MOE nucleosides.

44. The compound of claim 42 or 43 comprising two 3′-terminal nucleosides.

45. The compound of any of claims 33 to 44, wherein the modified oligonucleotide comprises at least one modified internucleoside linkage.

46. The compound of any of claims 33 to 45, wherein each internucleoside linkage of the modified oligonucleotide is selected from a phosphorothioate internucleoside linkage and an unmodified, phosphate internucleoside linkage.

47. The compound of claim 45 or 46, wherein each of the 6-10 3′-most internucleoside linkages of the modified oligonucleotide is a phosphorothioate internucleoside linkage.

48. The compound of any of claims 45 to 47, wherein the 5′-most internucleoside linkage of the modified oligonucleotide is a phosphorothioate internucleoside linkage.

49. The compound of any of claims 45 to 48, wherein the modified oligonucleotide comprises a region of at least 4 internucleoside linkages that alternate between phosphorothioate and phosphate internucleoside linkages.

50. The compound of any of claims 1 to 49, wherein the single-stranded oligonucleotide has two mismatches relative to the target region of the mRNA transcript.

51. The compound of any of claims 1 to 49, wherein the single-stranded oligonucleotide has three mismatches relative to the target region of the mRNA transcript.

52. The compound of any of claims 1 to 49, wherein the single-stranded oligonucleotide has four mismatches relative to the target region of the mRNA transcript.

53. The compound of any of claims 1 to 49, wherein the single-stranded oligonucleotide has five mismatches relative to the target region of the mRNA transcript.

54. The compound of any of claims 1 to 49, wherein the single-stranded oligonucleotide has six mismatches relative to the target region of the mRNA transcript.

55. The compound of any of claims 50 to 54, wherein the 5′-most nucleobase and 3′-most nucleobase of the single-stranded oligonucleotide are mismatches relative to the target region of the mRNA transcript.

56. The compound of any of claims 51 to 54, wherein the 5′-most nucleobase and the two 3′-most nucleobases of the single-stranded oligonucleotide are mismatches relative to the target region of the mRNA transcript.

57. The compound of any of claims 50 to 54, wherein the nucleobases at positions 9 and 14 of the single-stranded oligonucleotide are mismatches relative to the target region of the mRNA transcript.

58. The compound of any of claims 50 to 54, wherein the nucleobases at positions 9, 10, and 11 of the single-stranded oligonucleotide are mismatches relative to the target region of the mRNA transcript.

59. The compound of any of claims 1 to 58, wherein the complementary region is between the 5′-most nucleoside and the two 3′-most nucleosides of the single-stranded oligonucleotide and is 100% complementary to the target region of the mRNA transcript.

60. The compound of any of claims 1 to 58, wherein the complementary region is between the 5′-most nucleoside and the two 3′-most nucleosides of the single-stranded oligonucleotide and is 90% complementary to the target region of the mRNA transcript.

61. The compound of any of claims 1 to 58, wherein the complementary region is between the 5′-most nucleoside and the two 3′-most nucleosides of the single-stranded oligonucleotide and is 80% complementary to the target region of the mRNA transcript.

62. The compound of any of claims 1 to 61, wherein each of the nucleobases in the complementary region of the single-stranded oligonucleotide is selected from among adenine, guanine, cytosine, 5′-methylcytosine, thymine, and uracil.

63. The compound of any of claims 1 to 61, wherein each of the nucleobases in the single-stranded oligonucleotide is selected from among adenine, guanine, cytosine, 5′-methylcytosine, thymine, and uracil.

64. The compound of any of claims 1 to 63, wherein the single-stranded oligonucleotide does not comprise a modified nucleobase.

65. The compound of any of claims 1 to 63, wherein the single-stranded oligonucleotide comprises 5-methylcytosine.

66. The compound of any of claims 11 to 65, wherein the phosphorus moiety is an unmodified phosphate.

67. The compound of any of claims 11 to 65, wherein the phosphorus moiety is a 5′-(E)-vinylphosphonate group having the formula:

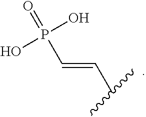 [SMILES: */C=C/P(=O)(O)O]

68. The compound of any of claims 1 to 67, wherein the compound comprises an unlocked nucleic acid or an abasic nucleoside.

69. The compound of claim 68, wherein the compound comprises an unlocked nucleic acid.

70. The compound of claim 69, wherein the nucleobase attached to the sugar moiety of the unlocked nucleic acid is mismatched relative to the corresponding nucleobase of the target region of the mRNA transcript.

71. The compound of any of claims 1 to 70, wherein the compound consists essentially of the single-stranded oligonucleotide.

72. The compound of claim 71, wherein the compound consists of the single-stranded oligonucleotide.

73. A pharmaceutical composition comprising the compound of any of claims 1 to 72, and a pharmaceutically acceptable carrier or diluent.

74. A method of treating a subject with epileptic encephalopathy or a neurodevelopmental disorder comprising contacting a cell with the compound or composition of any of claims 1 to 73.

75. The method of claim 74, wherein the cell is in vitro.

76. The method of claim 74, wherein the cell is in an animal, such as a human

77. The method of claim 76, wherein the contacting comprises delivery into the cerebrospinal fluid (CFS).

78. The method of any one of claims 74 to 77, wherein contacting comprises multiple administration of the compound or composition.

79. The method of any one of claims 77, further comprising assessing expression of a target protein in CSF of said animal following contacting.

80. The method of any one of claims 76 to 79, wherein the animal or human is an infant or child.

81. The method of any one of claims 74 to 80, wherein the expression of a protein encoded by a target mRNA is increased approximately 2-fold as compared to pretreatment levels.

82. Use of the compound or composition of any of claims 1 to 73 for the manufacture of a medicament for treating epileptic encephalopathy or a neurodevelopmental disorder.

83. Use of the compound or composition of any of claims 1 to 73 for treating epileptic encephalopathy or a neurodevelopmental disorder.


SEQUENCE LISTING (69 sequences)

SEQ ID NO: 1 - RNA, 37 nt, Homo sapiens
ucaccccacagaaacugcuggacacacugaagaaacu

SEQ ID NO: 2 - RNA, 38 nt, Homo sapiens
ugagcacaccauuugugcugcugcuguugucgugaaau

SEQ ID NO: 3 - RNA, 37 nt, Homo sapiens
guuugaaaguacugaagcacaaacauauaucaucucu

SEQ ID NO: 4 - RNA, 38 nt, Homo sapiens
gucugucuugaaacuuguuuaccuuaaaauuaucagaa

SEQ ID NO: 5 - RNA, 37 nt, Homo sapiens
uuugaaaucuccccuugcacugagauuagucgucaga

SEQ ID NO: 6 - RNA, 38 nt, Homo sapiens
ccaaagaaacaaagauccacacacacuccucaccccac

SEQ ID NO: 7 - DNA, 20 bp, Homo sapiens
cacacatcctcaccccacag

SEQ ID NO: 8 - DNA, 20 bp, Homo sapiens
accccacagaaactgctgga

SEQ ID NO: 9 - DNA, 20 bp, Homo sapiens
cagaaactgctggacacact

SEQ ID NO: 10 - DNA, 20 bp, Homo sapiens
ccatttgtgctgctgctgtt

SEQ ID NO: 11 - DNA, 20 bp, Homo sapiens
ttgtgctgctgctgttgtcg

SEQ ID NO: 12 - DNA, 20 bp, Homo sapiens
ctgctgctgttgtcgtgaaa

SEQ ID NO: 13 - DNA, 20 bp, Homo sapiens
gaaagtactgaagcacaaac

SEQ ID NO: 14 - DNA, 20 bp, Homo sapiens
gcacaaacatatatcatctc

SEQ ID NO: 15 - DNA, 20 bp, Homo sapiens
atcatctctgtaccattctg

SEQ ID NO: 16 - RNA, 20 nt, Homo sapiens
gucugucuugaaacuuguuu

SEQ ID NO: 17 - RNA, 20 nt, Homo sapiens
ucuugaaacuuguuuaccuu

SEQ ID NO: 18 - RNA, 20 nt, Homo sapiens
aaacuuguuuaccuuaaaau

SEQ ID NO: 19 - RNA, 20 nt, Homo sapiens
uguuuaccuuaaaauuauca

SEQ ID NO: 20 - RNA, 20 nt, Homo sapiens
uuugaaaucuccccuugcac

SEQ ID NO: 21 - RNA, 20 nt, Homo sapiens
aaucuccccuugcacugaga

SEQ ID NO: 22 - RNA, 20 nt, Homo sapiens
ccccuugcacugagauuagu

SEQ ID NO: 23 - RNA, 20 nt, Homo sapiens
ugcacugagauuagucguca

SEQ ID NO: 24 - RNA, 20 nt, Homo sapiens
ccaaagaaacaaagauccac

SEQ ID NO: 25 - RNA, 20 nt, Homo sapiens
gaaacaaagauccacacaca

SEQ ID NO: 26 - RNA, 20 nt, Homo sapiens
aaagauccacacacacuccu

SEQ ID NO: 27 - RNA, 20 nt, Homo sapiens
uccacacacacuccucaccc

SEQ ID NO: 28 - DNA, 20 bp, Artificial sequence
  Synthetic oligonucleotide
ctgtggggtgaggatgtgtg

SEQ ID NO: 29 - DNA, 20 bp, Artificial sequence
  Synthetic oligonucleotide
tccagcagtttctgtggggt

SEQ ID NO: 30 - DNA, 20 bp, Artificial sequence
  Synthetic oligonucleotide
agtgtgtccagcagtttctg

SEQ ID NO: 31 - DNA, 20 bp, Artificial sequence
  Synthetic oligonucleotide
aacagcagcagcacaaatgg

SEQ ID NO: 32 - DNA, 20 bp, Artificial sequence
  Synthetic oligonucleotide
cgacaacagcagcagcacaa

SEQ ID NO: 33 - DNA, 20 bp, Artificial sequence
  Synthetic oligonucleotide
tttcacgacaacagcagcag

SEQ ID NO: 34 - DNA, 20 bp, Artificial sequence
  Synthetic oligonucleotide
gtttgtgcttcagtactttc

SEQ ID NO: 35 - DNA, 20 bp, Artificial sequence
  Synthetic oligonucleotide
gagatgatatatgtttgtgc

SEQ ID NO: 36 - DNA, 20 bp, Artificial sequence
  Synthetic oligonucleotide
cagaatggtacagagatgat

SEQ ID NO: 37 - DNA, 20 bp, Artificial sequence
  Synthetic oligonucleotide
aaacaagtttcaagacagac

SEQ ID NO: 38 - DNA, 20 bp, Artificial sequence
  Synthetic oligonucleotide
aaggtaaacaagtttcaaga

SEQ ID NO: 39 - DNA, 20 bp, Artificial sequence
  Synthetic oligonucleotide
attttaaggtaaacaagttt

SEQ ID NO: 40 - DNA, 20 bp, Artificial sequence
  Synthetic oligonucleotide
tgataattttaaggtaaaca

SEQ ID NO: 41 - DNA, 20 bp, Artificial sequence
  Synthetic oligonucleotide
gtgcaaggggagatttcaaa

SEQ ID NO: 42 - DNA, 20 bp, Artificial sequence
  Synthetic oligonucleotide
tctcagtgcaaggggagatt

SEQ ID NO: 43 - DNA, 20 bp, Artificial sequence
  Synthetic oligonucleotide
actaatctcagtgcaagggg

SEQ ID NO: 44 - DNA, 20 bp, Artificial sequence
  Synthetic oligonucleotide
tgacgactaatctcagtgca

SEQ ID NO: 45 - DNA, 20 bp, Artificial sequence
  Synthetic oligonucleotide
gtggatctttgtttctttgg

SEQ ID NO: 46 - DNA, 20 bp, Artificial sequence
  Synthetic oligonucleotide
tgtgtgtggatctttgtttc

SEQ ID NO: 47 - DNA, 20 bp, Artificial sequence
  Synthetic oligonucleotide
aggagtgtgtgtggatcttt

SEQ ID NO: 48 - DNA, 20 bp, Artificial sequence
  Synthetic oligonucleotide
gggtgaggagtgtgtgtgga

SEQ ID NO: 49 - RNA, 20 nt, Artificial sequence
  Synthetic oligonucleotide
cuguggggugaggaugugug

SEQ ID NO: 50 - RNA, 20 nt, Artificial sequence
  Synthetic oligonucleotide
uccagcaguuucuguggggu

SEQ ID NO: 51 - RNA, 20 nt, Artificial sequence
  Synthetic oligonucleotide
aguguguccagcaguuucug

SEQ ID NO: 52 - RNA, 20 nt, Artificial sequence
  Synthetic oligonucleotide
aacagcagcagcacaaaugg

SEQ ID NO: 53 - RNA, 20 nt, Artificial sequence
  Synthetic oligonucleotide
uuucacgacaacagcagcag

SEQ ID NO: 54 - RNA, 20 nt, Artificial sequence
  Synthetic oligonucleotide
guuugugcuucaguacuuuc

SEQ ID NO: 55 - RNA, 20 nt, Artificial sequence
  Synthetic oligonucleotide
gagaugauauauguuugugc

SEQ ID NO: 56 - RNA, 20 nt, Artificial sequence
  Synthetic oligonucleotide
cagaaugguacagagaugau

SEQ ID NO: 57 - RNA, 20 nt, Artificial sequence
  Synthetic oligonucleotide
aaacaaguuucaagacagac

SEQ ID NO: 58 - RNA, 20 nt, Artificial sequence
  Synthetic oligonucleotide
aagguaaacaaguuucaaga

SEQ ID NO: 59 - RNA, 20 nt, Artificial sequence
  Synthetic oligonucleotide
auuuuaagguaaacaaguuu

SEQ ID NO: 60 - RNA, 20 nt, Artificial sequence
  Synthetic oligonucleotide
ugauaauuuuaagguaaaca

SEQ ID NO: 61 - RNA, 20 nt, Artificial sequence
  Synthetic oligonucleotide
gugcaaggggagauuucaaa

SEQ ID NO: 62 - RNA, 20 nt, Artificial sequence
  Synthetic oligonucleotide
ucucagugcaaggggagauu

SEQ ID NO: 63 - RNA, 20 nt, Artificial sequence
  Synthetic oligonucleotide
acuaaucucagugcaagggg

SEQ ID NO: 64 - RNA, 20 nt, Artificial sequence
  Synthetic oligonucleotide
ugacgacuaaucucagugca

SEQ ID NO: 65 - RNA, 20 nt, Artificial sequence
  Synthetic oligonucleotide
guggaucuuuguuucuuugg

SEQ ID NO: 66 - RNA, 20 nt, Artificial sequence
  Synthetic oligonucleotide
uguguguggaucuuuguuuc

SEQ ID NO: 67 - RNA, 20 nt, Artificial sequence
  Synthetic oligonucleotide
aggaguguguguggaucuuu

SEQ ID NO: 68 - RNA, 20 nt, Artificial sequence
  Synthetic oligonucleotide
gggugaggagugugugugga

SEQ ID NO: 69 - RNA, 21 nt, Homo sapiens
uguaccaaucuaguucguguu